SEQUENCE LISTING (19 sequences)

SEQ ID NO: 1 - DNA, 1137 bp, primate
  CDS (99)..(998)
  misc_feature (367): W; may be A or T
  mat_peptide (132)..(998)
cgcaggcggaccgggggcaaaggaggtggcatgtcggtcaggcacagcagggtcctgtgtccgcgctgagccgcgctctccctgctccagcaaggaccatgagggcgctggaggggccaggcctgtcgctgctgtgcctggtgttggcgctgcctgccctgctgccggtgccggctgtacgcggagtggcagaaacacccacctacccctggcgggacgcagagacaggggagcggctggtgtgcgcccagtgccccccaggcacctttgtgcagcggccgtgccgccgagacagccccatgacgtgtggcccgtgtccaccgcgccactacacgcagttctggaactacctggagcgctgccgctwctgctacgtcctctgcggggagcgtgaggaggaggcacgggcttgccacgccacccacaaccgtgcctgccgctgccgcaccggcttcttcgcgcacgctggtttctgcttggagcacgcatcgtgtccacctggtgccggcgtgattgccccgggcacccccagccagaacacgcagtgccagccgtgccccccaggcaccttctcagccagcagctccagctcagagcagtgccagccccaccgcaactgcacggccctgggcctggccctcaatgtgccaggctcttcctcccatgacaccctgtgcaccagctgcactggcttccccctcagcaccagggtaccaggagctgaggagtgtgagcgtgccgtcatcgactttgtggctttccaggacatctccatcaagaggctgcagcggctgctgcaggccctcgaggccccggagggctggggtccgacaccaagggcgggccgcgcggccttgcagctgaagctgcgtcggcggctcacggagctcctgggggcgcaggacggggcgctgctggtgcggctgctgcaggcgctgcgcgtggccaggatgcccgggctggagcggagcgtccgtgagcgcttcctccctgtgcactgatcctggccccctcttatttattctacatccttggcaccccacttgcactgaaagaggcttttttttaaatagaagaaatgaggtttcttaaagcttatttttataaagctttttcataaaaaaaaaaaaaaaaaaa

SEQ ID NO: 2 - protein, 300 aa, primate
  misc_feature (79): Xaa at residue 79 is undetermined.
MRALEGPGLSLLCLVLALPALLPVPAVRGVAETPTYPWRDAETGERLVCAQCPPGTFVQRPCRRDSPMTCGPCPPRHYTQFWNYLERCRXCYVLCGEREEEARACHATHNRACRCRTGFFAHAGFCLEHASCPPGAGVIAPGTPSQNTQCQPCPPGTFSASSSSSEQCQPHRNCTALGLALNVPGSSSHDTLCTSCTGFPLSTRVPGAEECERAVIDFVAFQDISIKRLQRLLQALEAPEGWGPTPRAGRAALQLKLRRRLTELLGAQDGALLVRLLQALRVARMPGLERSVRERFLPVH

SEQ ID NO: 3 - DNA, 1031 bp, primate
  CDS (402)..(1031)
  mat_peptide (525)..(1031)
  misc_feature (2): n; may be A, C, G, or T
  misc_feature (9): n; may be A, C, G, or T
  misc_feature (664): k; may be G or T
  misc_feature (956): n; may be A, C, G, or T
  misc_feature (989): n; may be A, C, G, or T
cngactcantccctcgccgaccagtctgggcagcggaggagggtggttggcagtggctggaagcttcgctatgggaagtcgttcctttgctctctcgcgcccagtcctcctccctggttctcctcagccgctgtcggaggagagcacccggagacgcgggctgcagtcgcggcggcttctccccgcctgggcggccgcgccgctgggcaggtgctgagcgcccctagagcctcccttgccgcctccctcctctgcccggccgcagcagtgcacatggggtgttggaggtagatgggctcccggcccgggaggcggcggtggatgcggcgctgggcagaagcagccgccgattccagctgccccgcgcgccccgggcgcccctgcgagtccccggttcagccatggggacctctccgagcagcagcaccgccctcgcctcctgcagccgcatcgcccgccgagccacagccacgatgatcgcgggctcccttctcctgcttggattccttagcaccaccacagctcagccagaacagaaggcctcgaatctcattggcacataccgccatgttgaccgtgccaccggccaggtgctaacctgtgacaagtgtccagcaggaacctatgtctctgagcattgtaccaacacaagctgcgcgtctgkcagcagttgccctgtggggacctttaccaggcatgagaatggcatagagaaatgccatgactgtagtcagccatgcccatggccaatgattgagaaattaccttgtgctgccttgactgaccgagaatgcacttgcccacctggcatgttccagtctaacgctacctgtgccccccatacggtgtgtcctgtgggttggggtgtgcggaagaaagggacagagactgaggatgtgcggtgtaagcagtgtgctcgggggtacttctcagatgtgccttctagtgtgatgaangcaaagcatacacagactgtctggatcagaacntggttggtgatcaagccggggggaccaaggagacagacaact

SEQ ID NO: 4 - protein, 210 aa, primate
  misc_feature (47): Xaa at residue 47 is undetermined.
  misc_feature (144): Xaa at residue 144 is undetermined.
  misc_feature (155): Xaa at residue 155 is undetermined.
MGTSPSSSTALASCSRIARRATATMIAGSLLLLGFLSTTTAQPEQKASNLIGTYRHVDRATGQVLTCDKCPAGTYVSEHCTNTSCASXSSCPVGTFTRHENGIEKCHDCSQPCPWPMIEKLPCAALTDRECTCPPGMFQSNATCAPHTVCPVGWGVRKKGTETEDVRCKQCARGYFSDVPSSVMXAKHTQTVWIRXWLVIKPGGPRRQTT

SEQ ID NO: 5 - DNA, 2877 bp, primate
  CDS (410)..(2374)
  mat_peptide (533)..(2374)
ggcacgagccgactcagtccctcgccgaccagtctgggcagcggaggagggtggttggcagtggctggaagcttcgctatgggaagtcgttcctttgctctctcgcgcccagtcctcctccctggttctcctcagccgctgtcggaggagagcacccggagacgcgggctgcagtcgcggcggcttctccccgcctgggcggccgcgccgctgggcaggtgctgagcgcccctagcgcctcccttgccgcctccctcctctgcccggccgcagcagtgcacatggggtgttggaggtagatgggctcccggcccgggaggcggcggtggatgcggcgctgggcagaagcagccgccgattccagctgccccgcgcgccccgggcgcccctgcgagtccccggttcagccatggggacctctccgagcagcagcaccgccctcgcctcctgcagccgcatcgcccgccgagccacagccacgatgatcgcgggctcccttctcctgcttggattccttagcaccaccacagctcagccagaacagaaggcctcgaatctcattggcacataccgccatgttgaccgtgccaccggccaggtgctaacctgtgacaagtgtccagcaggaacctatgtctctgagcattgtaccaacacaagcctgcgcgtctgcagcagttgccctgtggggacctttaccaggcatgagaatggcatagagaaatgccatgactgtagtcagccatgcccatggccaatgattgagaaattaccttgtgctgccttgactgaccgagaatgcacttgcccacctggcatgttccagtctaacgctacctgtgccccccatacggtgtgtcctgtgggttggggtgtgcggaagaaagggacagagactgaggatgtgcggtgtaagcagtgtgctcggggtaccttctcagatgtgccttctagtgtgatgaaatgcaaagcatacacagactgtctgagtcagaacctggtggtgatcaagccggggaccaaggagacagacaacgtctgtggcacactcccgtccttctccagctccacctcaccttcccctggcacagccatctttccacgccctgagcacatggaaacccatgaagtcccttcctccacttatgttcccaaaggcatgaactcaacagaatccaactcttctgcctctgttagaccaaaggtactgagtagcatccaggaagggacagtccctgacaacacaagctcagcaagggggaaggaagacgtgaacaagaccctcccaaaccttcaggtagtcaaccaccagcaaggcccccaccacagacacatcctgaagctgctgccgtccatggaggccactgggggcgagaagtccagcacgcccatcaagggccccaagaggggacatcctagacagaacctacacaagcattttgacatcaatgagcatttgccctggatgattgtgcttttcctgctgctggtgcttgtggtgattgtggtgtgcagtatccggaaaagctcgaggactctgaaaaaggggccccggcaggatcccagtgccattgtggaaaaggcagggctgaagaaatccatgactccaacccagaaccgggagaaatggatctactactgcaatggccatggtatcgatatcctgaagcttgtagcagcccaagtgggaagccagtggaaagatatctatcagtttctttgcaatgccagtgagagggaggttgctgctttctccaatgggtacacagccgaccacgagcgggcctacgcagctctgcagcactggaccatccggggccccgaggccagcctcgcccagctaattagcgccctgcgccagcaccggagaaacgatgttgtggagaagattcgtgggctgatggaagacaccacccagctggaaactgacaaactagctctcccgatgagccccagcccgcttagcccgagccccatccccagccccaacgcgaaacttgagaattccgctctcctgacggtggagccttccccacaggacaagaacaagggcttcttcgtggatgagtcggagccccttctccgctgtgactctacatccagcggctcctccgcgctgagcaggaacggttcctttattaccaaagaaaagaaggacacagtgttgcggcaggtacgcctggacccctgtgacttgcagcctatctttgatgacatgctccactttctaaatcctgaggagctgcgggtgattgaagagattccccaggctgaggacaaactagaccggctattcgaaattattggagtcaagagccaggaagccagccagaccctcctggactctgtttatagccatcttcctgacctgctgtagaacatagggatactgcattctggaaattactcaatttagtggcagggtggttttttaatttccttctgtgtctgatttttgttgtttggggtgtgtgtgtgtgtttgtgtgtgtgtgtgtgtgtgtgtgtgtgtgtgtttaacagagaatatggccagtgcttgagttctttctccttctctctctctcttttttttttaaataactcttctgggaagttggtttataagcctttgccaggtgtaactgttgtgaaatacccaccactaaagttttttaagttccatattttctccattttgccttcttatgtattttcaagattattctgtgcactttaaatttactcaacttaccataaatgcagtgtgacttttcccacacactggattgtgaggctcttaacttcttaaaagtataatggcatcttgtgaatcctataagcagtctttatgtctcttaacattcacacctactttttaaaaacaaatattattact

SEQ ID NO: 6 - protein, 655 aa, primate
MGTSPSSSTALASCSRIARRATATMIAGSLLLLGFLSTTTAQPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHLPWMIVLFLLLVLVVIVVCSIRKSSRTLKKGPRQDPSAIVEKAGLKKSMTPTQNREKWIYYCNGHGIDILKLVAAQVGSQWKDIYQFLCNASEREVAAFSNGYTADHERAYAALQHWTIRGPEASLAQLISALRQHRRNDVVEKIRGLMEDTTQLETDKLALPMSPSPLSPSPIPSPNAKLENSALLTVEPSPQDKNKGFFVDESEPLLRCDSTSSGSSALSRNGSFITKEKKDTVLRQVRLDPCDLQPIFDDMLHFLNPEELRVIEEIPQAEDKLDRLFEIIGVKSQEASQTLLDSVYSHLPDLL

SEQ ID NO: 7 - DNA, 1474 bp, primate
  CDS (1)..(1332)
  mat_peptide (124)..(1332)
atggggacctctccgagcagcagcaccgccctcgcctcctgcagccgcatcgcccgccgagccacagccacgatgatcgcgggctcccttctcctgcttggattccttagcaccaccacagctcagccagaacagaaggcctcgaatctcattggcacataccgccatgttgaccgtgccaccggccaggtgctaacctgtgacaagtgtccagcaggaacctatgtctctgagcattgtaccaacacaagcctgcgcgtctgcagcagttgccctgtggggacctttaccaggcatgagaatggcatagagaaatgccatgactgtagtcagccatgcccatggccaatgattgagaaattaccttgtgctgccttgactgaccgagaatgcacttgcccacctggcatgttccagtctaacgctacctgtgccccccatacggtgtgtcctgtgggttggggtgtgcggaagaaagggacagagactgaggatgtgcggtgtaagcagtgtgctcggggtaccttctcagatgtgccttctagtgtgatgaaatgcaaagcatacacagactgtctgagtcagaacctggtggtgatcaagccggggaccaaggagacagacaacgtctgtggcacactcccgtccttctccagctccacctcaccttcccctggcacagccatctttccacgccctgagcacatggaaacccatgaagtcccttcctccacttatgttcccaaaggcatgaactcaacagaatccaactcttctgcctctgttagaccaaaggtactgagtagcatccaggaagggacagtccctgacaacacaagctcagcaagggggaaggaagacgtgaacaagaccctcccaaaccttcaggtagtcaaccaccagcaaggcccccaccacagacacatcctgaagctgctgccgtccatggaggccactgggggcgagaagtccagcacgcccatcaagggccccaagaggggacatcctagacagaacctacacaagcattttgacatcaatgagcatttgccctggatgattgtgcttttcctgctgctggtgcttgtggtgattgtggtgtgcagtatccggaaaagctcgaggactctgaaaaaggggccccggcaggatcccagtgccattgtggaaaaggcagggctgaagaaatccatgactccaacccagaaccgggagaaatggatctactactgcaatggccatggaccccatgatgaggagtgggggttgatggagagacatattcaagatatttatattcaaagaagcaatcaagattcagaaagatggggttgataatttttacttcaccctgggaggcagcatagtgcagtgaaaggtatcgatatcctgaagcttgtagcagcccaagtgggaagccagtggaaagatatctatcagtttctttgcaatgccagtgagagggaggttgctg

SEQ ID NO: 8 - protein, 444 aa, primate
MGTSPSSSTALASCSRIARRATATMIAGSLLLLGFLSTTTAQPEQKASNLIGTYRHVDRATGQVLTCDKCPAGTYVSEHCTNTSLRVCSSCPVGTFTRHENGIEKCHDCSQPCPWPMIEKLPCAALTDRECTCPPGMFQSNATCAPHTVCPVGWGVRKKGTETEDVRCKQCARGTFSDVPSSVMKCKAYTDCLSQNLVVIKPGTKETDNVCGTLPSFSSSTSPSPGTAIFPRPEHMETHEVPSSTYVPKGMNSTESNSSASVRPKVLSSIQEGTVPDNTSSARGKEDVNKTLPNLQVVNHQQGPHHRHILKLLPSMEATGGEKSSTPIKGPKRGHPRQNLHKHFDINEHLPWMIVLFLLLVLVVIVVCSIRKSSRTLKKGPRQDPSAIVEKAGLKKSMTPTQNREKWIYYCNGHGPHDEEWGLMERHIQDIYIQRSNQDSERWG

SEQ ID NO: 9 - protein, 227 aa, rodent
MAPAALWVALVFELQLWATGHTVPAQVVLTPYKPEPGYECQISQEYYDRKAQMCCAKCPPGQYVKHFCNKTSDTVCADCEASMYTQVWNQFRTCLSCSSSCTTDQVEIRACTKQQNRVCACEAGRYCALKTHSGSCRQCMRLSKCGPGFGVASSRAPNGNVLCKACAPGTFSDTTSSTDVCRPHRICSILAIPGNASTDAVCAPESPTLSAIPRTLYVSQPEPTRSQ

SEQ ID NO: 10 - protein, 225 aa, primate
MAPVAVWAALAVGLELWAAAHALPAQVAFTPYAPEPGSTCRLREYYDQTAQMCCSKCSPGQHAKVFCTKTSDTVCDSCEDSTYTQLWNWVPECLSCGSRCSSDQVETQACTREQNRICTCRPGWYCALSKQEGCRLCAPLRKCRPGFGVARPGTETSDVVCKPCAPGTFSNTTSSTDICRPHQICNVVAIPGNASMDAVCTSTSPTRSMAPGAVHLPQPVSTRSQ

SEQ ID NO: 11 - protein, 187 aa, primate
MNKLLCCALVFLDISIKWTTQETFPPKYLHYDEETSHQLLCDKCPPGTYLKQHCTAKWKTVCAPCPDHYYTDSWHTSDECLYCSPVCKELQYVKQECNRTHNRVCECKEGRYLEIEFCLKHRSCPPGFGVVQAGTPERNTVCKRCPDGFFSNETSSKAPCRKHTNCSVFGLLLTQKGNATHDNICSG

SEQ ID NO: 12 - DNA, 636 bp, rodent
  CDS (104)..(553)
  mat_peptide (191)..(553)
ggcacgagggcgtttggcgcggaagtgctaccaagctgcggaaagcgtgagtctggagcacagcactggcgagtagcaggaataaacacgtttggtgagagccatggcactcaaggtcctacctctacacaggacggtgctcttcgctgccattctcttcctactccacctggcatgtaaagtgagttgcgaaaccggagattgcaggcagcaggaattcaaggatcgatctggaaactgtgtcctctgcaaacagtgcggacctggcatggagttgtccaaggaatgtggcttcggctatggggaggatgcacagtgtgtgccctgcaggccgcaccggttcaaggaagactggggtttccagaagtgtaagccatgtgcggactgtgcgctggtgaaccgctttcagagggccaactgctcacacaccagtgatgctgtctgcggggactgcctgccaggattttaccggaagaccaaactggttggttttcaagacatggagtgtgtgccctgcggagacccacctcctccctacgaaccacactgtgagtgatgtgccaagtggcagcagacctttaaaaaaaaaagaaaaaaaaacaaacaaaaacaaaaaaaaaaaaaaaaaaaaaaaaa

SEQ ID NO: 13 - protein, 150 aa, rodent
MALKVLPLHRTVLFAAILFLLHLACKVSCETGDCRQQEFKDRSGNCVLCKQCGPGMELSKECGFGYGEDAQCVPCRPHRFKEDWGFQKCKPCADCALVNRFQRANCSHTSDAVCGDCLPGFYRKTKLVGFQDMECVPCGDPPPPYEPHCE

SEQ ID NO: 14 - DNA, 474 bp, primate
  CDS (78)..(473)
  misc_feature (308): n; may be A, C, G, or T
  misc_feature (315): n; may be A, C, G, or T
  misc_feature (333): n; may be A, C, G, or T
  misc_feature (412): n; may be A, C, G, or T
  misc_feature (431): n; may be A, C, G, or T
  misc_feature (436): n; may be A, C, G, or T
  misc_feature (444): n; may be A, C, G, or T
  misc_feature (473): n; may be A, C, G, or T
cgcgctgaggtggatttgtaccggagtcccatttgggagcaagagccatctactcgtccgttaccggccttcccaccatggattgccaagaaaatgagtactgggaccaatggggacggtgtgtcacctgccaacggtgtggtcctggacaggagctatccaaggattgtggttatggagagggtggagatgcctactgcacagcctgccctcctcgcagtacaaaagcagctggggccaccacaaatgtcagagttgcatcacctgtgctgtcatcaatcgtgttcagaaggttcaactgcacagtnacctctnatgctgtctgtggggganggtttgcccaagtttctaaccgaaagacacgccattggaaggctgccaggaccaaggatggcatcccgtggcacaaagncagacccccaacttctganggttncaaagtgnctttccaattggagcttaatgggaggcana

SEQ ID NO: 15 - protein, 132 aa, primate
  misc_feature (77): Xaa at residue 77 is undetermined.
  misc_feature (80): Xaa at residue 80 is undetermined.
  misc_feature (86): Xaa at residue 86 is undetermined.
  misc_feature (112): Xaa at residue 112 is undetermined.
  misc_feature (118): Xaa at residue 118 is undetermined.
  misc_feature (120): Xaa at residue 120 is undetermined.
  misc_feature (123): Xaa at residue 123 is undetermined.
  misc_feature (132): Xaa at residue 132 is undetermined.
MDCQENEYWDQWGRCVTCQRCGPGQELSKDCGYGEGGDAYCTACPPRSTKAAGATTNVRVASPVLSSIVFRRFNCTXTSXAVCGGXFAQVSNRKTRHWKAARTKDGIPWHKXRPPTSXGXKVXFQLELNGRX

SEQ ID NO: 16 - DNA, 546 bp, primate
  CDS (78)..(308)
  misc_feature (317): n; may be A, C, G, or T
  misc_feature (340): n; may be A, C, G, or T
  misc_feature (351): n; may be A, C, G, or T
  misc_feature (389): n; may be A, C, G, or T
  misc_feature (398): n; may be A, C, G, or T
  misc_feature (428): n; may be A, C, G, or T
  misc_feature (429): n; may be A, C, G, or T
  misc_feature (433): n; may be A, C, G, or T
  misc_feature (452): n; may be A, C, G, or T
  misc_feature (468): n; may be A, C, G, or T
  misc_feature (483): n; may be A, C, G, or T
  misc_feature (534): n; may be A, C, G, or T
  misc_feature (541): n; may be A, C, G, or T
cgcgctgaggtggatttgtaccggagtcccatttgggagcaagagccatctactcgtccgttaccggccttcccaccatggattgccaagaaaatgagtactgggaccaatggggacggtgtgtcacctgccaacggtgtggtcctggacaggagctatccaaggattgtggttatggagagggtggagatgcctactgcacagcctgccctcctcgcaggtacaaaagcagctggggccaccacaaatgtcagagttgcatcacctgtgctgtcatcaatcgtgttcagaaggtccaactgcacagctaacctctnatgctgtctgtggggatgtttgncccaagttctnaccgaaaagacacgccatgggaaggctggcaggaccangaatggccntcccgtggcagaaagccagaccccccaacnnctgnaggttccaatgtggccttnccatttggaagcttantgggaaggcagatgncaacccaaagtggccccttcagggaggccaaaatttgttggcaatgggtgnagcagcntgcca

SEQ ID NO: 17 - protein, 77 aa, primate
MDCQENEYWDQWGRCVTCQRCGPGQELSKDCGYGEGGDAYCTACPPRRYKSSWGHHKCQSCITCAVINRVQKVQLHS

SEQ ID NO: 18 - DNA, 932 bp, primate
  CDS (78)..(770)
  misc_feature (782): n; may be A, C, G, or T
cgcgctgaggtggatttgtaccggagtcccatttgggagcaagagccatctactcgtccgttaccggccttcccaccatggattgccaagaaaatgagtactgggaccaatggggacggtgtgtcacctgccaacggtgtggtcctggacaggagctatccaaggattgtggttatggagagggtggagatgcctactgcacagcctgccctcctcgcaggtacaaaagcagctggggccaccacaaatgtcagagttgcatcacctgtgctgtcatcaatcgtgttcagaaggtcaactgcacagctacctctaatgctgtctgtggggactgtttgcccaggttctaccgaaagacacgcattggaggcctgcaggaccaagagtgcatcccgtgcacgaagcagacccccacctctgaggttcaatgtgccttccagttgagcttagtggaggcagatgcacccacagtgccccctcaggaggccacacttgttgcactggtgagcagcctgctagtggtgtttaccctggccttcctggggctcttcttcctctactgcaagcagttcttcaacagacattgccagcgtggaggtttgctgcagtttgaggctgataaaacagcaaaggaggaatctctcttccccgtgccacccagcaaggagaccagtgctgagtcccaagtctcttgggcccctggcagccttgcccagttgttctctctggactctgttcctataccacaacagcagcaggggcctgaaatgtgatgtccacangagctaataccctacagatggggcatatcctatcccatcccaccagaggattgattctccatttcacaaggactgatctggagcatttcttgcttccctgttgtagtctggggagccagattccacattcatgggactaccagacatgtt

SEQ ID NO: 19 - protein, 231 aa, primate
MDCQENEYWDQWGRCVTCQRCGPGQELSKDCGYGEGGDAYCTACPPRRYKSSWGHHKCQSCITCAVINRVQKVNCTATSNAVCGDCLPRFYRKTRIGGLQDQECIPCTKQTPTSEVQCAFQLSLVEADAPTVPPQEATLVALVSSLLVVFTLAFLGLFFLYCKQFFNRHCQRGGLLQFEADKTAKEESLFPVPPSKETSAESQVSWAPGSLAQLFSLDSVPIPQQQQGPEM

(19) United States
(12) Patent Application Publication
(10) Pub. No.: US 2002/0143147 A1
(43) Pub. Date: Oct. 3, 2002

(54) Mammalian genes; related reagents

(76) Inventors: Erin E. Murphy, Palo Alto, CA (US); Jeanine D. Mattson, San Francisco, CA (US); Elizabeth Esther Mary Bates, Lyon (FR); Daniel M. Gorman, Newark, CA (US); Serge J.E. Lebecque, Civrieux d' Azergue (FR)

Correspondence Address: TOWNSEND AND TOWNSEND AND CREW, LLP, TWO EMBARCADERO CENTER, EIGHTH FLOOR, SAN FRANCISCO, CA, 94111-3834, US

(21) Appl. No.: 09/840,795

(22) Filed: Apr. 23, 2001

Related U.S. Application Data
(63) Continuation of application No. 09/351,777, filed on Jul. 12, 1999, now abandoned.
(60) Provisional application No. 60/099,999, filed on Sep. 11, 1998.
(60) Provisional application No. 60/093,897, filed on Jul. 23, 1998.
(60) Provisional application No. 60/092,658, filed on Jul. 13, 1998.

(51) Int. Cl. C07K 14/705; C07H 21/04; C12P 21/02; C12N 5/06

(52) U.S. Cl. 530/350; 536/23.5; 435/325; 435/69.1; 435/320.1

(57) ABSTRACT

Purified genes from a mammal, reagents related thereto including purified proteins, specific antibodies, and nucleic acids encoding th polypeptides are provided. Methods of using said reagents and diagnostic kits are also provided.

This filing is a conversion to a U.S. Utility Patent Application of U.S. Provisional Patent Application U.S. Ser. No. 60/092,658; U.S. Ser. No. 60/093,897; and U.S. Ser. No. 60/099,999; each of which is incorporated herein by reference.

### FIELD OF THE INVENTION

The present invention pertains to compositions related to proteins which exhibit sequence similarity to TNF receptors which function in controlling activation and expansion of mammalian cells, e.g., cells of a mammalian immune system. In particular, it provides purified genes, proteins, antibodies, and related reagents useful, e.g., to separate or identify particular cell types, or to regulate activation, development, differentiation, and function of various cell types, including hematopoietic cells.

### BACKGROUND OF THE INVENTION

The activation of resting T cells is critical to most immune responses and allows these cells to exert their regulatory or effector capabilities. See, e.g., Paul (ed. 1993) Fundamental Immunology 3d ed., Raven Press, N.Y. Increased adhesion between T cells and antigen presenting cells (APC) or other forms of primary stimuli, e.g., immobilized monoclonal antibodies (mAb), can potentiate the T-cell receptor signals. T-cell activation and T cell expansion depends upon engagement of the T-cell receptor (TCR) and co-stimulatory signals provided by accessory cells. See, e.g., Jenkins and Johnson (1993) Curr. Opin. Immunol. 5:361-367; Bierer and Hahn (1993) Semin. Immunol. 5:249-261; June, et al. (1990) Immunol. Today 11:211-216; and Jenkins (1994) Immunity 1:443-446. A major, and well-studied, co-stimulatory interaction for T cells involves either CD28 or CTLA-4 on T cells with either B7 or B70 (Jenkins (1994) Immunity 1:443-446). Recent studies on CD28 deficient mice (Shahinian, et al. (1993) Science 261:609-612; Green, et al. (1994) Immunity 1:501-508) and CTLA-4 immunoglobulin expressing transgenic mice (Ronchese, et al. (1994) J. Exp. Med. 179:809-817) have revealed deficiencies in some T-cell responses though these mice have normal primary immune responses and normal CTL responses to lymphocytic choriomeningitis virus and vesicular stomatitis virus. As a result, both these studies conclude that other co-stimulatory molecules must be supporting T-cell function. However, identification of these molecules which mediate distinct costimulatory signals has been difficult.

Tumor Necrosis Factor (TNF) is the prototypic member of an emerging family of cytokines that function as prominent mediators of immune regulation and the inflammatory response. These ligands are typically type II membrane proteins, with homology at the carboxy terminus. A proteolytic processed soluble protein often is produced. See, e.g., Smith, et al. (1994) Cell 76-959-962; Armitage (1994) Current Opinion in Immunology 6:407-413; Gruss and Dower (1995) Blood 85:3378-3404; Wiley, et al. (1995) Immunity 3:673-682; and Baker and Reddy (1996) Oncogene 12:1-9. Crucial roles for these family members are evidenced by a number of studies, and they are implicated in regulation of apoptosis, peripheral tolerance, Ig maturation and isotype switching, and general B cell and T cell functions. See, e.g., Thomson (ed. 1994) The Cytokine Handbook Academic Press, San Diego, Calif.; Naismith and Sprang (1998) Trends Biochem. Sci. 23:74-79; Lucas, et al. (1997) J. Leukoc. Biol. 61:551-558; Reddi (1997) Cell 89:159-161; Van Deventer (1997) Gut 40:443-448; Jablonska (1997) Postepy. Hig. Med. Dosw. 51:567-575; Hill and Lunec (1996) Mol. Aspects Med. 17:455-509; Aderka (1996) Cytokine Growth Factor Rev. 7:231-240; Lotz, et al. (1996) J. Leukoc. Biol. 60:1-7; and Gruss and Dower (1995) Cytokines Mol. Ther. 1:75-105. These imply fundamental roles in immune and developmental networks relevant to human therapeutic needs. The identification of ligands and cell surface receptors allow determination of pairs, which will be useful in modulating such signal transduction.

The discovery of new cell markers is always potentially useful. Moreover, the inability to modulate activation signals prevents control of inappropriate developmental or physiological responses in the immune system. The present invention provides at least one alternative costimulatory molecule, which will be useful as a marker for cell types, and agonists and antagonists of which will be useful in modulating a plethora of immune conditions or responses.

### SUMMARY OF THE INVENTION

The present invention is based, in part, upon the discovery of genes which encode proteins which exhibit sequence homology to receptors for TNF ligands. It provides a gene encoding a 300 amino acid protein, designated HDTEA84; another encoding a 210 amino acid polypeptide, presumably a fragment, designated HSLJD37R; and another designated RANKL (RANK-Like; see Anderson, et al. (1997) Nature 390:175-179). Each gene exhibits similarity to receptors for TNF, CD40, osteoprotegerin, and viral forms of TNF receptors. Each gene is represented by a primate, e.g., human, embodiment, which description thereby enables mammalian genes, proteins, antibodies, and uses thereof. Functional equivalents exhibiting significant sequence homology are available from other mammalian, e.g., rodent, and other species.

More particularly, the present invention provides a substantially pure or recombinant HDTEA84, HSLJD37R, or RANKL protein or peptide fragment thereof. Various embodiments include a protein or peptide selected from a protein or peptide from a warm blooded animal selected from the group of birds and mammals, including a primate or. rodent; a protein or peptide comprising at least one polypeptide segment of SEQ ID NO: 2 or SEQ ID NO: 4, 6, or 8 or SEQ ID NO: 13, 15, 17, or 19; a polypeptide which exhibits a post-translational modification pattern distinct from natural HDTEA84, HSLJD37R, or RANKL; or a polypeptide which binds specifically to a polyclonal antibody preparation selected for specificity of binding to any of the proteins. The protein or peptide can comprise a sequence from the HDTEA84, the HSLJD37R, or RANKL; or be a fusion protein. The invention further provides a composition of matter selected from: a substantially pure or recombinant mature, e.g., signal processed form of, HDTEA84, HSLJD37R, or RANKL polypeptide exhibiting identity over a length of at least about 12 amino acids to SEQ ID NO: 2, SEQ ID NO: 4, 6, or 8, or SEQ ID NO: 13, 15, 17, or 19; a natural sequence HDTEA84 of SEQ ID NO: 2, HSLJD37R of SEQ ID NO: 4, 6, or 8, or RANKL of SEQ ID NO: 13, 15, 17, or 19; or a fusion protein comprising HDTEA84, HSLJD37R, or RANKL sequence. In certain preferred embodiments, the substantially pure or isolated protein comprising a segment exhibiting sequence identity over specified lengths to a corresponding portion of an HDTEA84, HSLJD37R, or RANKL. Other embodiments include, e.g., the composition of matter described, wherein said: HDTEA84 comprises a mature sequence of Table 1; or polypeptide: is from a warm blooded animal selected from a mammal, including a primate; comprises at least one polypeptide segment of SEQ ID NO: 2; HSLJD37R comprises a mature sequence of Table 2; or polypeptide: is from a warm blooded animal selected from a mammal, including a primate; comprises at least one polypeptide segment of SEQ ID NO: 4, 6, or 8; RANKL comprises a mature sequence of Table 4; or polypeptide: is from a warm blooded animal selected from a mammal, including a primate; comprises at least one polypeptide segment of SEQ ID NO: 13, 15, 17, or 19; exhibits a plurality of portions exhibiting said identity; is a natural allelic variant of HDTEA84, HSLJD37R, or RANKL; has a length at least about 30 amino acids; exhibits at least two non-overlapping epitopes which are specific for a mammalian HDTEA84, HSLJD37R, or RANKL; exhibits at least two non-overlapping epitopes which are specific for a primate HDTEA84; exhibits at least two non-overlapping epitopes which are specific for a primate HSLJD37R; exhibits at least two non-overlapping epitopes which are specific for a primate RANKL; is not glycosylated; is a synthetic polypeptide; is attached to a solid substrate; is conjugated to another chemical moiety; is a 5-fold or less substitution from natural sequence; or is a deletion or insertion variant from a natural sequence. Other embodiments include a composition comprising: a sterile HDTEA84, HSLJD37R, or RANKL protein or peptide; or the HDTEA84, HSLJD37R, or RANKL protein or peptide and a carrier, wherein said carrier is: an aqueous compound, including water, saline, and/or buffer; and/or formulated for oral, rectal, nasal, topical, or parenteral administration. Fusion protein forms include those comprising: mature protein comprising sequence of Table 1; mature protein comprising sequence of Table 2; mature protein comprising sequence of Table 4; a detection or purification tag, including a FLAG, His6, or Ig sequence; or sequence of another TNF antagonist. Kits include, e.g., those comprising said protein or polypeptide, and: a compartment comprising said protein or polypeptide; and/or instructions for use or disposal of reagents in said kit.

Another embodiment is a composition comprising an HDTEA84, HSLJD37R, or RANKL polypeptide and a pharmaceutically acceptable carrier. Other compositions may combine said entities with an agonist or antagonist of other T cell signaling molecules, e.g., signaling entities through the T cell receptor, CD40, CD40 ligand, CTLA-8, CD28, B7, B70, BAS-1, SLAM, etc.

The invention also embraces an antibody which specifically binds an HDTEA84, HSLJD37R, or RANKL polypeptide, e.g., wherein the polypeptide is from a primate, including a human; the antibody is raised against a purified HDTEA84 polypeptide sequence of SEQ ID NO: 2; the antibody is raised against a purified HSLJD37R polypeptide sequence of SEQ ID NO: 4, 6, or 8; the antibody is raised against a purified RANKL polypeptide sequence of SEQ ID NO: 13, 15, 17, or 19; the antibody is a monoclonal antibody; or the antibody is labeled. Other binding compounds are provided, e.g., comprising an antigen binding portion from an antibody, which specifically binds to a natural HDTEA84, HSLJD37R, or RANKL polypeptide, wherein: said polypeptide is a primate polypeptide; said binding compound is an Fv, Fab, or Fab2 fragment; said binding compound is conjugated to another chemical moiety; or said antibody: is raised against a peptide sequence of a mature polypeptide comprising sequence of Table 1, 2, or 4; is raised against a mature HDTEA84, HSLJD37R, or RANKL; is raised to a purified HDTEA84, HSLJD37R, or RANKL; is immunoselected; is a polyclonal antibody; binds to a denatured HDTEA84, HSLJD37R, or RANKL; exhibits a Kd to antigen of at least 30 μM; is attached to a solid substrate, including a bead or plastic membrane; is in a sterile composition; or is detectably labeled, including a radioactive or fluorescent label. Kits include, e.g., those comprising said binding compound, and: a compartment comprising said binding compound; and/or instructions for use or disposal of reagents in said kit.

Such binding compositions also provide methods of purifying an HDTEA84, HSLJD37R, or RANKL polypeptide from other materials in a mixture comprising contacting said mixture to an antibody, and separating bound HDTEA84, HSLJD37R, or RANKL from other materials;

Certain other compositions include those comprising: a sterile binding compound, or said binding compound and a carrier, wherein said carrier is: an aqueous compound, including water, saline, and/or buffer; and/or formulated for oral, rectal, nasal, topical, or parenteral administration.

Another aspect of the invention is an isolated or recombinant nucleic acid capable of encoding an HDTEA84, HSLJD37R, or RANKL protein or peptide, including a nucleic acid which encodes a sequence of signal processed SEQ ID NO: 2, or 4, 6, or 8, or 13, 15, 17, or 19; which includes a coding sequence of SEQ ID NO: 1, or 3, 5, or 7, or 12, 14, 16, or 18; or which encodes a sequence from an extracellular domain of a natural HDTEA84, HSLJD37R, or RANKL. Such nucleic acid embodiments also include an expression or replicating vector. Various other nucleic acid embodiments are provided, e.g., an isolated or recombinant nucleic acid encoding said protein or peptide or fusion protein, wherein: said TNF receptor family protein is from a mammal, including a primate; or said nucleic acid: encodes an antigenic peptide sequence of Table 1, of Table 2, or of Table 4; encodes a plurality of antigenic peptide sequences of Table 1, of Table 2, or of Table 4; exhibits identity to a natural cDNA encoding said segment; is an expression vector; further comprises an origin of replication; is from a natural source; comprises a detectable label; comprises synthetic nucleotide sequence; is less than 6 kb, preferably less than 3 kb; is from a mammal, including a primate; comprises a natural full length coding sequence; is a hybridization probe for a gene encoding said TNF ligand family protein; or is a PCR primer, PCR product, or mutagenesis primer. The invention also provides a cell or tissue comprising such a recombinant nucleic acid, e.g., wherein said cell is: a prokaryotic cell; a eukaryotic cell; a bacterial cell; a yeast cell; an insect cell; a mammalian cell; a mouse cell; a primate cell; or a human cell.

Also provided are a method of expressing an HDTEA84, HSLJD37R, or RANKL peptide by expressing a nucleic acid encoding said polypeptide, preferably signal processed forms. The invention also provides a cell, tissue, organ, or organism comprising a nucleic acid encoding a such peptide.

Kit embodiments include those, e.g., which comprise said nucleic acid and: a compartment further comprising an HDTEA84 protein or polypeptide; and/or instructions for use or disposal of reagents in said kit.

The invention further provides a nucleic acid which: hybridizes under wash conditions of 40° C. and less than 500 mM salt to the coding portion of SEQ ID NO: 1, of SEQ ID NO: 3, 5, or 7, or of SEQ ID NO: 12, 14, 16, or 18; or exhibits identity over a stretch of at least about 30 nucleotides to a primate HDTEA84, HSLJD37R, or RANKL, including a human. In other embodiments, the nucleic acid hybridizes where the nucleic acid, wherein: said wash conditions are at 55° C. and/or 400 mM salt; or exhibiting identity over at least 40 nucleotides. In yet other embodiments, the nucleic acid hybridizes, wherein: said wash conditions are at 65° C. and/or 200 mM salt; or exhibiting identity over at least 50 nucleotides.

The invention also provides a kit containing a substantially pure HDTEA84, HSLJD37R, or RANKL or fragment; an antibody or receptor which specifically binds an HDTEA84, HSLJD37R, or RANKL; or a nucleic acid, or its complement, encoding an HDTEA84, HSLJD37R, or RANKL polypeptide. This kit also provides methods for detecting in a sample the presence of a nucleic acid, protein, or antibody, comprising testing said sample with such a kit.

The invention also supplies methods of modulating the physiology of a cell comprising contacting said cell with a substantially pure HDTEA84, HSLJD37R, or RANKL polypeptide; an antibody or binding partner which specifically binds an HDTEA84, HSLJD37R, or RANKL; or a nucleic acid encoding an HDTEA84, HSLJD37R, or RANKL polypeptide. Certain preferred embodiments include a method where the cell is a precursor cell and the modulating of physiology is proliferation or induction of development; or where the cell is in a tissue and/or in an organism.

Another method provided is treating an organism having an abnormal immune response by administering to said organism an effective dose of: an antibody or binding partner which binds specifically to an HDTEA84, HSLJD37R, or RANKL; a substantially pure HDTEA84, HSLJD37R, or RANKL polypeptide; or a nucleic acid encoding an HDTEA84, HSLJD37R, or RANKL polypeptide.

### DETAILED DESCRIPTION OF THE PREFERRED EMBODIMENTS

All references cited herein-are incorporated herein by reference to the same extent as if each individual publication or patent application was specifically and individually indicated to be incorporated by reference.

Outline

I. General

II. Purified Receptors

A. physical properties

B. biological properties

III. Physical Variants

A. sequence variants, fragments

B. post-translational variants

1. glycosylation

2. others

IV. Functional Variants

A. analogs, fragments

1. agonists

2. antagonists

B. mimetics

1. protein

2. chemicals

C. species variants

V. Antibodies

A. polyclonal

B. monoclonal

C. fragments, binding compositions

VI. Nucleic Acids

A. natural isolates; methods

B. synthetic genes

C. methods to isolate

VII. Making Receptors, mimetics

A. recombinant methods

B. synthetic methods

C. natural purification

VIII. Uses

A. diagnostic

B. therapeutic

IX. Kits

A. nucleic acid reagents

B. protein reagents

C. antibody reagents

X. Isolating a binding partner (ligand)

I. General

The present invention provides amino acid sequences and DNA sequences encoding various mammalian proteins, e.g., which are polypeptides produced by selected cells. Among these proteins are those which modulate or mediate, e.g., induce or prevent proliferation or differentiation of, interacting cells. HDTEA84, HSLJD37R, and RANKL genes and proteins are also provided, which are related to the TNF signaling pathways. The antigens HDTEA84, HSLJD37R, and RANKL, and fragments, or antagonists will be useful in physiological modulation of cells expressing receptors for, e.g., ligands of the TNF family. Some of these antigens are forms which appear to lack a membrane spanning segment, suggesting that they are soluble forms of receptor. This suggests that the soluble proteins can serve as antagonists of the TNF-like ligands. In addition, it is likely that membrane spanning forms exist, which serve as signaling receptors mediating cellular response to the ligands.

The HDTEA84 gene has been detected in cDNA libraries derived form Hodgkin's lymphoma, endothelial cells, keratinocytes, prostrate, and cerebellum. It exhibits significant sequence similarity to the osteoprotegerin ligand receptor reported by Lacey, et al. (1998) Cell 93:165-176. The HDTEA84 will likely modulate proliferation or development by antagonizing its respective ligand. Membrane associated forms should exist, likely alternatively spliced transcription products.

The HSLJD37R exhibits like similarity to receptors for TNF. While the first embodiment is an incomplete sequence, the available portion currently lacks an identified transmembrane segment. Additional efforts provide a full length sequence, and an alternative splice variant.

The rodent 427152#4 Rank-like (RANKL) was detected in a rodent cDNA library panel probed with Mouse 427152#4 (204 bp). Signals were detected in CH12 (B cell line); rag-1 thymus; rag-1 heart; rag-1 brain (best signal); rag-1 testes; rag-1 liver; normal lung; rag-1 lung; asthmatic lung; tolerized and challenged lung; Nippo-infected lung; Nippo IL-4 K.O. lung; Nippo anti-IL-5 treated lung; influenza lung; guinea pig allergic lung; w.t. stomach; and w.t. colon on a 3 day exposure at −80° C. with an intensifier screen. On a 2 week exposure at −80° C. with screen, signals were also detected in the following libraries: Mel 14+ naive; Mell4+ Th1; Mel 14+ Th2; Th1 3 week Bl/6; large B cell; bEnd3+TNFα+IL-10, guinea pig normal lung; and Rag Hh- colon.

The primate Rank-like homologs of rodent 427152#4 were detected in a human cDNA library panel probed with Mouse 427152#4 (204 bp). Signals were detected in Monkey asthma lung 4 h (1.6-2.0 kb) and adult placenta (2.5-3.0 kb) on a 3 day exposure at −80° C. with screen. On a 2 week exposure at −80° C. with screen, signals were also detected in the following libraries: CD1a+ 95% DC activated CHA (kidney epithelial carcinoma cell line); Monkey lung normal; Psoriasis skin; fetal lung; fetal ovary; fetal testes; and fetal spleen.

Each of these proteins will also be useful as antigens, e.g., immunogens, for raising antibodies to various epitopes on the protein, linear and/or conformational epitopes. The molecules may be useful in defining various cell subsets, either by the molecules produced by, or by expression of membrane forms of the receptors. Such cells should be responsive to the respective ligands. Soluble forms of the receptors should serve as antagonists of the ligand, binding to the ligand and preventing interaction with membrane forms, which would mediate signaling.

Each gene expresses polypeptides which exhibit structural motifs characteristic of a member of the TNF receptor family. Table 1 provides the nucleic acid and predicted amino acid sequences for primate, e.g., human, HDTEA84. Table 2 provides the nucleic acid and predicted amino acid sequences for primate, e.g., human, HSLJD37R. Table 3 shows a polypeptide sequence comparison of various members of the TNF receptor family. Table 4 provides the nucleic acid and predicted amino acid sequences for rodent, e.g., mouse, and primate, e.g., human, RANKL.

TABLE 1

Primate, e.g., human, HDTEA84 nucleotide sequence (SEQ ID
NO: 1), with an ORF (SEQ ID NO: 2) running from about nucleotides 99
to 998. Nucleotide W at position 367 may also be A or T. Predicted
signal cleavage site is indicated.

| | |
|---|---|
| cgcaggcgga ccgggggcaa aggaggtggc atgtcggtca ggcacagcag ggtcctgtgt | 60 |
| ccgcgctgag ccgcgctctc cctgctccag caaggacc atg agg gcg ctg gag ggg | 116 |
| Met Arg Ala Leu Glu Gly | |
| −10 | |
| cca ggc ctg tcg ctg ctg tgc ctg gtg ttg gcg ctg cct gcc ctg ctg | 164 |
| Pro Gly Leu Ser Leu Leu Cys Leu Val Leu Ala Leu Pro Ala Leu Leu | |
| −5 −1 1 5 10 | |
| ccg gtg ccg gct gta cgc gga gtg gca gaa aca ccc acc tac ccc tgg | 212 |
| Pro Val Pro Ala Val Arg Gly Val Ala Glu Thr Pro Thr Tyr Pro Trp | |
| 15 20 25 | |
| cgg gac gca gag aca ggg gag cgg ctg gtg tgc gcc cag tgc ccc cca | 260 |
| Arg Asp Ala Glu Thr Gly Glu Arg Leu Val Cys Ala Gln Cys Pro Pro | |
| 30 35 40 | |
| ggc acc ttt gtg cag cgg ccg tgc cgc cga gac agc ccc atg acg tgt | 308 |
| Gly Thr Phe Val Gln Arg Pro Cys Arg Arg Asp Ser Pro Met Thr Cys | |
| 45 50 55 | |
| ggc ccg tgt cca ccg cgc cac tac acg cag ttc tgg aac tac ctg gag | 356 |
| Gly Pro Cys Pro Pro Arg His Tyr Thr Gln Phe Trp Asn Tyr Leu Glu | |
| 60 65 70 75 | |
| cgc tgc cgc twc tgc tac gtc ctc tgc ggg gag cgt gag gag gag gca | 404 |
| Arg Cys Arg Xaa Cys Tyr Val Leu Cys Gly Glu Arg Glu Glu Glu Ala | |
| 80 85 90 | |
| cgg gct tgc cac gcc acc cac aac cgt gcc tgc cgc tgc cgc acc ggc | 452 |
| Arg Ala Cys His Ala Thr His Asn Arg Ala Cys Arg Cys Arg Thr Gly | |
| 95 100 105 | |
| ttc ttc gcg cac gct ggt ttc tgc ttg gag cac gca tcg tgt cca cct | 500 |
| Phe Phe Ala His Ala Gly Phe Cys Leu Glu His Ala Ser Cys Pro Pro | |
| 110 115 120 | |
| ggt gcc ggc gtg att gcc ccg ggc acc ccc agc cag aac acg cag tgc | 548 |
| Gly Ala Gly Val Ile Ala Pro Gly Thr Pro Ser Gln Asn Thr Gln Cys | |
| 125 130 135 | |
| cag ccg tgc ccc cca ggc acc ttc tca gcc agc agc tcc agc tca gag | 596 |
| Gln Pro Cys Pro Pro Gly Thr Phe Ser Ala Ser Ser Ser Ser Ser Glu | |
| 140 145 150 155 | |
| cag tgc cag ccc cac cgc aac tgc acg gcc ctg ggc ctg gcc ctc aat | 644 |
| Gln Cys Gln Pro His Arg Asn Cys Thr Ala Leu Gly Leu Ala Leu Asn | |
| 160 165 170 | |
| gtg cca ggc tct tcc tcc cat gac acc ctg tgc acc agc tgc act ggc | 692 |
| Val Pro Gly Ser Ser Ser His Asp Thr Leu Cys Thr Ser Cys Thr Gly | |
| 175 180 185 | |
| ttc ccc ctc agc acc agg gta cca gga gct gag gag tgt gag cgt gcc | 740 |
| Phe Pro Leu Ser Thr Arg Val Pro Gly Ala Glu Glu Cys Glu Arg Ala | |
| 190 195 200 | |
| gtc atc gac ttt gtg gct ttc cag gac atc tcc atc aag agg ctg cag | 788 |
| Val Ile Asp Phe Val Ala Phe Gln Asp Ile Ser Ile Lys Arg Leu Gln | |
| 205 210 215 | |
| cgg ctg ctg cag gcc ctc gag gcc ccg gag ggc tgg ggt ccg aca cca | 836 |
| Arg Leu Leu Gln Ala Leu Glu Ala Pro Glu Gly Trp Gly Pro Thr Pro | |
| 220 225 230 235 | |
| agg gcg ggc cgc gcg gcc ttg cag ctg aag ctg cgt cgg cgg ctc acg | 884 |
| Arg Ala Gly Arg Ala Ala Leu Gln Leu Lys Leu Arg Arg Arg Leu Thr | |
| 240 245 250 | |
| gag ctc ctg ggg gcg cag gac ggg gcg ctg ctg gtg cgg ctg ctg cag | 932 |
| Glu Leu Leu Gly Ala Gln Asp Gly Ala Leu Leu Val Arg Leu Leu Gln | |
| 255 260 265 | |
| gcg ctg cgc gtg gcc agg atg ccc ggg ctg gag cgg agc gtc cgt gag | 980 |
| Ala Leu Arg Val Ala Arg Met Pro Gly Leu Glu Arg Ser Val Arg Glu | |
| 270 275 280 | |
| cgc ttc ctc cct gtg cac tgatcctggc cccctcttat ttattctaca | 1028 |
| Arg Phe Leu Pro Val His | |
| 285 | |
| tccttggcac cccacttgca ctgaaagagg ctttttttta aatagaagaa atgaggtttc | 1088 |
| ttaaagctta tttttataaa gctttttcat aaaaaaaaaa aaaaaaaaa | 1137 |
| MRALEGPGLS LLCLVLALPA LLPVPAVRGV AETPTYPWRD AETGERLVCA QCPPGTFVQR | |
| PCRRDSPMTC GPCPPRHYTQ FWNYLERCR. CYVLCGEREE EARACHATHN RACRCRTGFF | |
| AHAGFCLEHA SCPPGAGVIA PGTPSQNTQC QPCPPGTFSA SSSSSEQCQP HRNCTALGLA | |
| LNVPGSSSHD TLCTSCTGFP LSTRVPGAEE CERAVIDFVA FQDISIKRLQ RLLQALEAPE | |
| GWGPTPRAGR AALQLKLRRR LTELLGAQDG ALLVRLLQAL RVARMPGLER SVRERFLPVH | |

TABLE 2

Partial primate, e.g., human, HSLJD37R (SEQ ID NO: 3 and
4) . Nucleotides 2, 956, and 989 designated N, each may be A, C, G,
or T; and nucleotide 664 designated K, may be G or T. See also
Genbank sequences N49208, AA991608, AA918818, and AA837291.

| | |
|---|---|
| cngactcant ccctcgccga ccagtctggg cagcggagga gggtggttgg cagtggctgg | 60 |
| aagcttcgct atgggaagtc gttcctttgc tctctcgcgc ccagtcctcc tccctggttc | 120 |
| tcctcagccg ctgtcggagg agagcacccg gagacgcggg ctgcagtcgc ggcggcttct | 180 |
| ccccgcctgg gcggccgcgc cgctgggcag gtgctgagcg cccctagagc ctcccttgcc | 240 |
| gcctccctcc tctgcccggc cgcagcagtg cacatggggt gttggaggta gatgggctcc | 300 |
| cggcccggga ggcggcggtg gatgcggcgc tgggcagaag cagccgccga ttccagctgc | 360 |
| cccgcgcgcc ccgggcgccc ctgcgagtcc ccggttcagc c atg ggg acc tct ccg | 416 |
| Met Gly Thr Ser Pro | |
| −40 | |
| agc agc agc acc gcc ctc gcc tcc tgc agc cgc atc gcc cgc cga gcc | 464 |
| Ser Ser Ser Thr Ala Leu Ala Ser Cys Ser Arg Ile Ala Arg Arg Ala | |
| −35 −30 −25 | |
| aca gcc acg atg atc gcg ggc tcc ctt ctc ctg ctt gga ttc ctt agc | 512 |
| Thr Ala Thr Met Ile Ala Gly Ser Leu Leu Leu Leu Gly Phe Leu Ser | |
| −20 −15 −10 −5 | |
| acc acc aca gct cag cca gaa cag aag gcc tcg aat ctc att ggc aca | 560 |
| Thr Thr Thr Ala Gln Pro Glu Gln Lys Ala Ser Asn Leu Ile Gly Thr | |
| −1 1 5 10 | |
| tac cgc cat gtt gac cgt gcc acc ggc cag gtg cta acc tgt gac aag | 608 |
| Tyr Arg His Val Asp Arg Ala Thr Gly Gln Val Leu Thr Cys Asp Lys | |
| 15 20 25 | |
| tgt cca gca gga acc tat gtc tct gag cat tgt acc aac aca agc tgc | 656 |
| Cys Pro Ala Gly Thr Tyr Val Ser Glu His Cys Thr Asn Thr Ser Cys | |
| 30 35 40 | |
| gcg tct gkc agc agt tgc cct gtg ggg acc ttt acc agg cat gag aat | 704 |
| Ala Ser Xaa Ser Ser Cys Pro Val Gly Thr Phe Thr Arg His Glu Asn | |
| 45 50 55 60 | |
| ggc ata gag aaa tgc cat gac tgt agt cag cca tgc cca tgg cca atg | 752 |
| Gly Ile Glu Lys Cys His Asp Cys Ser Gln Pro Cys Pro Trp Pro Met | |
| 65 70 75 | |
| att gag aaa tta cct tgt gct gcc ttg act gac cga gaa tgc act tgc | 800 |
| Ile Glu Lys Leu Pro Cys Ala Ala Leu Thr Asp Arg Glu Cys Thr Cys | |
| 80 85 90 | |
| cca cct ggc atg ttc cag tct aac gct acc tgt gcc ccc cat acg gtg | 848 |
| Pro Pro Gly Met Phe Gln Ser Asn Ala Thr Cys Ala Pro His Thr Val | |
| 95 100 105 | |
| tgt cct gtg ggt tgg ggt gtg cgg aag aaa ggg aca gag act gag gat | 896 |
| Cys Pro Val Gly Trp Gly Val Arg Lys Lys Gly Thr Glu Thr Glu Asp | |
| 110 115 120 | |
| gtg cgg tgt aag cag tgt gct cgg ggg tac ttc tca gat gtg cct tct | 944 |
| Val Arg Cys Lys Gln Cys Ala Arg Gly Tyr Phe Ser Asp Val Pro Ser | |
| 125 130 135 140 | |
| agt gtg atg aan gca aag cat aca cag act gtc tgg atc aga acn tgg | 992 |
| Ser Val Met Xaa Ala Lys His Thr Gln Thr Val Trp Ile Arg Xaa Trp | |
| 145 150 155 | |
| ttg gtg atc aag ccg ggg gga cca agg aga cag aca act | 1031 |
| Leu Val Ile Lys Pro Gly Gly Pro Arg Arg Gln Thr Thr | |
| 160 165 | |
| MGTSPSSSTA LASCSRIARR ATATMIAGSL LLLGFLSTTT AQPEQKASNL IGTYRHVDRA | |
| TGQVLTCDKC PAGTYVSEHC TNTSCASxSS CPVGTFTRHE NGIEKCHDCS QPCPWPMIEK | |
| LPCAALTDRE CTCPPGMFQS NATCAPHTVC PVGWGVRKKG TETEDVRCKQ CARGYFSDVP | |
| SSVMxAKHTQ TVWIRxWLVI KPGGPRRQTT | |
| supplemented sequence, with a predicted transmembrane segment from | |
| about leu309 to arg330 (SEQ ID NO: 5 and 6): | |
| ggcacgagcc gactcagtcc ctcgccgacc agtctgggca gcggaggagg gtggttggca | 60 |
| gtggctggaa gcttcgctat gggaagtcgt tcctttgctc tctcgcgccc agtcctcctc | 120 |
| cctggttctc ctcagccgct gtcggaggag agcacccgga gacgcgggct gcagtcgcgg | 180 |
| cggcttctcc ccgcctgggc ggccgcgccg ctgggcaggt gctgagcgcc cctagcgcct | 240 |
| cccttgccgc ctccctcctc tgcccggccg cagcagtgca catggggtgt tggaggtaga | 300 |
| tgggctcccg gcccgggagg cggcggtgga tgcggcgctg ggcagaagca gccgccgatt | 360 |
| ccagctgccc cgcgcgcccc gggcgcccct gcgagtcccc ggttcagcc atg ggg acc | 418 |
| Met Gly Thr | |
| −40 | |
| tct ccg agc agc agc acc gcc ctc gcc tcc tgc agc cgc atc gcc cgc | 466 |
| Ser Pro Ser Ser Ser Thr Ala Leu Ala Ser Cys Ser Arg Ile Ala Arg | |
| −35 −30 −25 | |
| cga gcc aca gcc acg atg atc gcg ggc tcc ctt ctc ctg ctt gga ttc | 514 |
| Arg Ala Thr Ala Thr Met Ile Ala Gly Ser Leu Leu Leu Leu Gly Phe | |
| −20 −15 −10 | |
| ctt agc acc acc aca gct cag cca gaa cag aag gcc tcg aat ctc att | 562 |
| Leu Ser Thr Thr Thr Ala Gln Pro Glu Gln Lys Ala Ser Asn Leu Ile | |
| −5 −1 1 5 10 | |
| ggc aca tac cgc cat gtt gac cgt gcc acc ggc cag gtg cta acc tgt | 610 |
| Gly Thr Tyr Arg His Val Asp Arg Ala Thr Gly Gln Val Leu Thr Cys | |
| 15 20 25 | |
| gac aag tgt cca gca gga acc tat gtc tct gag cat tgt acc aac aca | 658 |
| Asp Lys Cys Pro Ala Gly Thr Tyr Val Ser Glu His Cys Thr Asn Thr | |
| 30 35 40 | |
| agc ctg cgc gtc tgc agc agt tgc cct gtg ggg acc ttt acc agg cat | 706 |
| Ser Leu Arg Val Cys Ser Ser Cys Pro Val Gly Thr Phe Thr Arg His | |
| 45 50 55 | |
| gag aat ggc ata gag aaa tgc cat gac tgt agt cag cca tgc cca tgg | 754 |
| Glu Asn Gly Ile Glu Lys Cys His Asp Cys Ser Gln Pro Cys Pro Trp | |
| 60 65 70 | |
| cca atg att gag aaa tta cct tgt gct gcc ttg act gac cga gaa tgc | 802 |
| Pro Met Ile Glu Lys Leu Pro Cys Ala Ala Leu Thr Asp Arg Glu Cys | |
| 75 80 85 90 | |
| act tgc cca cct ggc atg ttc cag tct aac gct acc tgt gcc ccc cat | 850 |
| Thr Cys Pro Pro Gly Met Phe Gln Ser Asn Ala Thr Cys Ala Pro His | |
| 95 100 105 | |
| acg gtg tgt cct gtg ggt tgg ggt gtg cgg aag aaa ggg aca gag act | 898 |
| Thr Val Cys Pro Val Gly Trp Gly Val Arg Lys Lys Gly Thr Glu Thr | |
| 110 115 120 | |
| gag gat gtg cgg tgt aag cag tgt gct cgg ggt acc ttc tca gat gtg | 946 |
| Glu Asp Val Arg Cys Lys Gln Cys Ala Arg Gly Thr Phe Ser Asp Val | |
| 125 130 135 | |
| cct tct agt gtg atg aaa tgc aaa gca tac aca gac tgt ctg agt cag | 994 |
| Pro Ser Ser Val Met Lys Cys Lys Ala Tyr Thr Asp Cys Leu Ser Gln | |
| 140 145 150 | |
| aac ctg gtg gtg atc aag ccg ggg acc aag gag aca gac aac gtc tgt | 1042 |
| Asn Leu Val Val Ile Lys Pro Gly Thr Lys Glu Thr Asp Asn Val Cys | |
| 155 160 165 170 | |
| ggc aca ctc ccg tcc ttc tcc agc tcc acc tca cct tcc cct ggc aca | 1090 |
| Gly Thr Leu Pro Ser Phe Ser Ser Ser Thr Ser Pro Ser Pro Gly Thr | |
| 175 180 185 | |
| gcc atc ttt cca cgc cct gag cac atg gaa acc cat gaa gtc cct tcc | 1138 |
| Ala Ile Phe Pro Arg Pro Glu His Met Glu Thr His Glu Val Pro Ser | |
| 190 195 200 | |
| tcc act tat gtt ccc aaa ggc atg aac tca aca gaa tcc aac tct tct | 1186 |
| Ser Thr Tyr Val Pro Lys Gly Met Asn Ser Thr Glu Ser Asn Ser Ser | |
| 205 210 215 | |
| gcc tct gtt aga cca aag gta ctg agt agc atc cag gaa ggg aca gtc | 1234 |
| Ala Ser Val Arg Pro Lys Val Leu Ser Ser Ile Gln Glu Gly Thr Val | |
| 220 225 230 | |
| cct gac aac aca agc tca gca agg ggg aag gaa gac gtg aac aag acc | 1282 |
| Pro Asp Asn Thr Ser Ser Ala Arg Gly Lys Glu Asp Val Asn Lys Thr | |
| 235 240 245 250 | |
| ctc cca aac ctt cag gta gtc aac cac cag caa ggc ccc cac cac aga | 1330 |
| Leu Pro Asn Leu Gln Val Val Asn His Gln Gln Gly Pro His His Arg | |
| 255 260 265 | |
| cac atc ctg aag ctg ctg ccg tcc atg gag gcc act ggg ggc gag aag | 1378 |
| His Ile Leu Lys Leu Leu Pro Ser Met Glu Ala Thr Gly Gly Glu Lys | |
| 270 275 280 | |
| tcc agc acg ccc atc aag ggc ccc aag agg gga cat cct aga cag aac | 1426 |
| Ser Ser Thr Pro Ile Lys Gly Pro Lys Arg Gly His Pro Arg Gln Asn | |
| 285 290 295 | |
| cta cac aag cat ttt gac atc aat gag cat ttg ccc tgg atg att gtg | 1474 |
| Leu His Lys His Phe Asp Ile Asn Glu His Leu Pro Trp Met Ile Val | |
| 300 305 310 | |
| ctt ttc ctg ctg ctg gtg ctt gtg gtg att gtg gtg tgc agt atc cgg | 1522 |
| Leu Phe Leu Leu Leu Val Leu Val Val Ile Val Val Cys Ser Ile Arg | |
| 315 320 325 330 | |
| aaa agc tcg agg act ctg aaa aag ggg ccc cgg cag gat ccc agt gcc | 1570 |
| Lys Ser Ser Arg Thr Leu Lys Lys Gly Pro Arg Gln Asp Pro Ser Ala | |
| 335 340 345 | |
| att gtg gaa aag gca ggg ctg aag aaa tcc atg act cca acc cag aac | 1618 |
| Ile Val Glu Lys Ala Gly Leu Lys Lys Ser Met Thr Pro Thr Gln Asn | |
| 350 355 360 | |
| cgg gag aaa tgg atc tac tac tgc aat ggc cat ggt atc gat atc ctg | 1666 |
| Arg Glu Lys Trp Ile Tyr Tyr Cys Asn Gly His Gly Ile Asp Ile Leu | |
| 365 370 375 | |
| aag ctt gta gca gcc caa gtg gga agc cag tgg aaa gat atc tat cag | 1714 |
| Lys Leu Val Ala Ala Gln Val Gly Ser Gln Trp Lys Asp Ile Tyr Gln | |
| 380 385 390 | |
| ttt ctt tgc aat gcc agt gag agg gag gtt gct gct ttc tcc aat ggg | 1762 |
| Phe Leu Cys Asn Ala Ser Glu Arg Glu Val Ala Ala Phe Ser Asn Gly | |
| 395 400 405 410 | |
| tac aca gcc gac cac gag cgg gcc tac gca gct ctg cag cac tgg acc | 1810 |
| Tyr Thr Ala Asp His Glu Arg Ala Tyr Ala Ala Leu Gln His Trp Thr | |
| 415 420 425 | |
| atc cgg ggc ccc gag gcc agc ctc gcc cag cta att agc gcc ctg cgc | 1858 |
| Ile Arg Gly Pro Glu Ala Ser Leu Ala Gln Leu Ile Ser Ala Leu Arg | |
| 430 435 440 | |
| cag cac cgg aga aac gat gtt gtg gag aag att cgt ggg ctg atg gaa | 1906 |
| Gln His Arg Arg Asn Asp Val Val Glu Lys Ile Arg Gly Leu Met Glu | |
| 445 450 455 | |
| gac acc acc cag ctg gaa act gac aaa cta gct ctc ccg atg agc ccc | 1954 |
| Asp Thr Thr Gln Leu Glu Thr Asp Lys Leu Ala Leu Pro Met Ser Pro | |
| 460 465 470 | |
| agc ccg ctt agc ccg agc ccc atc ccc agc ccc aac gcg aaa ctt gag | 2002 |
| Ser Pro Leu Ser Pro Ser Pro Ile Pro Ser Pro Asn Ala Lys Leu Glu | |
| 475 480 485 490 | |
| aat tcc gct ctc ctg acg gtg gag cct tcc cca cag gac aag aac aag | 2050 |
| Asn Ser Ala Leu Leu Thr Val Glu Pro Ser Pro Gln Asp Lys Asn Lys | |
| 495 500 505 | |
| ggc ttc ttc gtg gat gag tcg gag ccc ctt ctc cgc tgt gac tct aca | 2098 |
| Gly Phe Phe Val Asp Glu Ser Glu Pro Leu Leu Arg Cys Asp Ser Thr | |
| 510 515 520 | |
| tcc agc ggc tcc tcc gcg ctg agc agg aac ggt tcc ttt att acc aaa | 2146 |
| Ser Ser Gly Ser Ser Ala Leu Ser Arg Asn Gly Ser Phe Ile Thr Lys | |
| 525 530 535 | |
| gaa aag aag gac aca gtg ttg cgg cag gta cgc ctg gac ccc tgt gac | 2194 |
| Glu Lys Lys Asp Thr Val Leu Arg Gln Val Arg Leu Asp Pro Cys Asp | |
| 540 545 550 | |
| ttg cag cct atc ttt gat gac atg ctc cac ttt cta aat cct gag gag | 2242 |
| Leu Gln Pro Ile Phe Asp Asp Met Leu His Phe Leu Asn Pro Glu Glu | |
| 555 560 565 570 | |
| ctg cgg gtg att gaa gag att ccc cag gct gag gac aaa cta gac cgg | 2290 |
| Leu Arg Val Ile Glu Glu Ile Pro Gln Ala Glu Asp Lys Leu Asp Arg | |
| 575 580 585 | |
| cta ttc gaa att att gga gtc aag agc cag gaa gcc agc cag acc ctc | 2338 |
| Leu Phe Glu Ile Ile Gly Val Lys Ser Gln Glu Ala Ser Gln Thr Leu | |
| 590 595 600 | |
| ctg gac tct gtt tat agc cat ctt cct gac ctg ctg tagaacatag | 2384 |
| Leu Asp Ser Val Tyr Ser His Leu Pro Asp Leu Leu | |
| 605 610 | |
| ggatactgca ttctggaaat tactcaattt agtggcaggg tggtttttta atttccttct | 2444 |
| gtgtctgatt tttgttgttt ggggtgtgtg tgtgtgtttg tgtgtgtgtg tgtgtgtgtg | 2504 |
| tgtgtgtgtg tttaacagag aatatggcca gtgcttgagt tctttctcct tctctctctc | 2564 |
| tctttttttt ttaaataact cttctgggaa gttggtttat aagcctttgc caggtgtaac | 2624 |
| tgttgtgaaa tacccaccac taaagttttt taagttccat attttctcca ttttgccttc | 2684 |
| ttatgtattt tcaagattat tctgtgcact ttaaatttac tcaacttacc ataaatgcag | 2744 |
| tgtgactttt cccacacact ggattgtgag gctcttaact tcttaaaagt ataatggcat | 2804 |
| cttgtgaatc ctataagcag tctttatgtc tcttaacatt cacacctact ttttaaaaac | 2864 |
| aaatattatt act | 2877 |
| MGTSPSSSTA LASCSRIARR ATATMIAGSL LLLGFLSTTT AQPEQKASNL IGTYRHVDRA | |
| TGQVLTCDKC PAGTYVSEHC TNTSLRVCSS CPVGTFTRHE NGIEKCHDCS QPCPWPMIEK | |
| LPCAALTDRE CTCPPGMFQS NATCAPHTVC PVGWGVRKKG TETEDVRCKQ CARGTFSDVP | |
| SSVMKCKAYT DCLSQNLVVI KPGTKETDNV CGTLPSFSSS TSPSPGTAIF PRPEHMETHE | |
| VPSSTYVPKG MNSTESNSSA SVRPKVLSSI QEGTVPDNTS SARGKEDVNK TLPNLQVVNH | |
| QQGPHHRHIL KLLPSMEATG GEKSSTPIKG PKRGHPRQNL HKHFDINEHL PWMIVLFLLL | |
| VLVVIVVCSI RKSSRTLKKG PRQDPSAIVE KAGLKKSMTP TQNREKWIYY CNGHGIDILK | |
| LVAAQVGSQW KDIYQFLCNA SEREVAAFSN GYTADHERAY AALQHWTIRG PEASLAQLIS | |
| ALRQHRRNDV VEKIRGLMED TTQLETDKLA LPMSPSPLSP SPIPSPNAKL ENSALLTVEP | |
| SPQDKNKGFF VDESEPLLRC DSTSSGSSAL SRNGSFITKE KKDTVLRQVR LDPCDLQPIF | |
| DDMLHFLNPE ELRVIEEIPQ AEDKLDRLFE IIGVKSQEAS QTLLDSVYSH LPDLL | |
| alternatively spliced variant results from insertion of another | |
| segment of sequence after nucleotide 1653 of SEQ ID NO: 5 (SEQ ID NO: | |
| 7 and 8): | |
| atg ggg acc tct ccg agc agc agc acc gcc ctc gcc tcc tgc agc cgc | 48 |
| Met Gly Thr Ser Pro Ser Ser Ser Thr Ala Leu Ala Ser Cys Ser Arg | |
| −40 −35 −30 | |
| atc gcc cgc cga gcc aca gcc acg atg atc gcg ggc tcc ctt ctc ctg | 96 |
| Ile Ala Arg Arg Ala Thr Ala Thr Met Ile Ala Gly Ser Leu Leu Leu | |
| −25 −20 −15 −10 | |
| ctt gga ttc ctt agc acc acc aca gct cag cca gaa cag aag gcc tcg | 144 |
| Leu Gly Phe Leu Ser Thr Thr Thr Ala Gln Pro Glu Gln Lys Ala Ser | |
| −5 −1 1 5 | |
| aat ctc att ggc aca tac cgc cat gtt gac cgt gcc acc ggc cag gtg | 192 |
| Asn Leu Ile Gly Thr Tyr Arg His Val Asp Arg Ala Thr Gly Gln Val | |
| 10 15 20 | |
| cta acc tgt gac aag tgt cca gca gga acc tat gtc tct gag cat tgt | 240 |
| Leu Thr Cys Asp Lys Cys Pro Ala Gly Thr Tyr Val Ser Glu His Cys | |
| 25 30 35 | |
| acc aac aca agc ctg cgc gtc tgc agc agt tgc cct gtg ggg acc ttt | 288 |
| Thr Asn Thr Ser Leu Arg Val Cys Ser Ser Cys Pro Val Gly Thr Phe | |
| 40 45 50 55 | |
| acc agg cat gag aat ggc ata gag aaa tgc cat gac tgt agt cag cca | 336 |
| Thr Arg His Glu Asn Gly Ile Glu Lys Cys His Asp Cys Ser Gln Pro | |
| 60 65 70 | |
| tgc cca tgg cca atg att gag aaa tta cct tgt gct gcc ttg act gac | 384 |
| Cys Pro Trp Pro Met Ile Glu Lys Leu Pro Cys Ala Ala Leu Thr Asp | |
| 75 80 85 | |
| cga gaa tgc act tgc cca cct ggc atg ttc cag tct aac gct acc tgt | 432 |
| Arg Glu Cys Thr Cys Pro Pro Gly Met Phe Gln Ser Asn Ala Thr Cys | |
| 90 95 100 | |
| gcc ccc cat acg gtg tgt cct gtg ggt tgg ggt gtg cgg aag aaa ggg | 480 |
| Ala Pro His Thr Val Cys Pro Val Gly Trp Gly Val Arg Lys Lys Gly | |
| 105 110 115 | |
| aca gag act gag gat gtg cgg tgt aag cag tgt gct cgg ggt acc ttc | 528 |
| Thr Glu Thr Glu Asp Val Arg Cys Lys Gln Cys Ala Arg Gly Thr Phe | |
| 120 125 130 135 | |
| tca gat gtg cct tct agt gtg atg aaa tgc aaa gca tac aca gac tgt | 576 |
| Ser Asp Val Pro Ser Ser Val Met Lys Cys Lys Ala Tyr Thr Asp Cys | |
| 140 145 150 | |
| ctg agt cag aac ctg gtg gtg atc aag ccg ggg acc aag gag aca gac | 624 |
| Leu Ser Gln Asn Leu Val Val Ile Lys Pro Gly Thr Lys Glu Thr Asp | |
| 155 160 165 | |
| aac gtc tgt ggc aca ctc ccg tcc ttc tcc agc tcc acc tca cct tcc | 672 |
| Asn Val Cys Gly Thr Leu Pro Ser Phe Ser Ser Ser Thr Ser Pro Ser | |
| 170 175 180 | |
| cct ggc aca gcc atc ttt cca cgc cct gag cac atg gaa acc cat gaa | 720 |
| Pro Gly Thr Ala Ile Phe Pro Arg Pro Glu His Met Glu Thr His Glu | |
| 185 190 195 | |
| gtc cct tcc tcc act tat gtt ccc aaa ggc atg aac tca aca gaa tcc | 768 |
| Val Pro Ser Ser Thr Tyr Val Pro Lys Gly Met Asn Ser Thr Glu Ser | |
| 200 205 210 215 | |
| aac tct tct gcc tct gtt aga cca aag gta ctg agt agc atc cag gaa | 816 |
| Asn Ser Ser Ala Ser Val Arg Pro Lys Val Leu Ser Ser Ile Gln Glu | |
| 220 225 230 | |
| ggg aca gtc cct gac aac aca agc tca gca agg ggg aag gaa gac gtg | 864 |
| Gly Thr Val Pro Asp Asn Thr Ser Ser Ala Arg Gly Lys Glu Asp Val | |
| 235 240 245 | |
| aac aag acc ctc cca aac ctt cag gta gtc aac cac cag caa ggc ccc | 912 |
| Asn Lys Thr Leu Pro Asn Leu Gln Val Val Asn His Gln Gln Gly Pro | |
| 250 255 260 | |
| cac cac aga cac atc ctg aag ctg ctg ccg tcc atg gag gcc act ggg | 960 |
| His His Arg His Ile Leu Lys Leu Leu Pro Ser Met Glu Ala Thr Gly | |
| 265 270 275 | |
| ggc gag aag tcc agc acg ccc atc aag ggc ccc aag agg gga cat cct | 1008 |
| Gly Glu Lys Ser Ser Thr Pro Ile Lys Gly Pro Lys Arg Gly His Pro | |
| 280 285 290 295 | |
| aga cag aac cta cac aag cat ttt gac atc aat gag cat ttg ccc tgg | 1056 |
| Arg Gln Asn Leu His Lys His Phe Asp Ile Asn Glu His Leu Pro Trp | |
| 300 305 310 | |
| atg att gtg ctt ttc ctg ctg ctg gtg ctt gtg gtg att gtg gtg tgc | 1104 |
| Met Ile Val Leu Phe Leu Leu Leu Val Leu Val Val Ile Val Val Cys | |
| 315 320 325 | |
| agt atc cgg aaa agc tcg agg act ctg aaa aag ggg ccc cgg cag gat | 1152 |
| Ser Ile Arg Lys Ser Ser Arg Thr Leu Lys Lys Gly Pro Arg Gln Asp | |
| 330 335 340 | |
| ccc agt gcc att gtg gaa aag gca ggg ctg aag aaa tcc atg act cca | 1200 |
| Pro Ser Ala Ile Val Glu Lys Ala Gly Leu Lys Lys Ser Met Thr Pro | |
| 345 350 355 | |
| acc cag aac cgg gag aaa tgg atc tac tac tgc aat ggc cat gga ccc | 1248 |
| Thr Gln Asn Arg Glu Lys Trp Ile Tyr Tyr Cys Asn Gly His Gly Pro | |
| 360 365 370 375 | |
| cat gat gag gag tgg ggg ttg atg gag aga cat att caa gat att tat | 1296 |
| His Asp Glu Glu Trp Gly Leu Met Glu Arg His Ile Gln Asp Ile Tyr | |
| 380 385 390 | |
| att caa aga agc aat caa gat tca gaa aga tgg ggt tgataatttt | 1342 |
| Ile Gln Arg Ser Asn Gln Asp Ser Glu Arg Trp Gly | |
| 395 400 | |
| tacttcaccc tgggaggcag catagtgcag tgaaaggtat cgatatcctg aagcttgtag | 1402 |
| cagcccaagt gggaagccag tggaaagata tctatcagtt tctttgcaat gccagtgaga | 1462 |
| gggaggttgc tg | 1474 |
| MGTSPSSSTA LASCSRIARR ATATMIAGSL LLLGFLSTTT AQPEQKASNL IGTYRHVDRA | |
| TGQVLTCDKC PAGTYVSEHC TNTSLRVCSS CPVGTFTRHE NGIEKCHDCS QPCPWPMIEK | |
| LPCAALTDRE CTCPPGMFQS NATCAPHTVC PVGWGVRKKG TETEDVRCKQ CARGTFSDVP | |
| SSVMKCKAYT DCLSQNLVVI KPGTKETDNV CGTLPSFSSS TSPSPGTAIF PRPEHMETHE | |
| VPSSTYVPKG MNSTESNSSA SVRPKVLSSI QEQTVPDNTS SARGKEDVNK TLPNLQVVNH | |
| QQGPHHRHIL KLLPSMEATG GEKSSTPIKG PKRGHPRQNL HKHFDINEHL PWMIVLFLLL | |
| VLVVIVVCSI RKSSRTLKKG PRQDPSAIVE KAGLKKSMTP TQNREKWIYY CNGHGPHDEE | |
| WGLMERHIQD IYIQRSNQDS ERWG | |

TABLE 3

Alignment of related TNF receptor family members. Murine TNF-R2
is SEQ ID NO: 9; human TNF-R2 is SEQ ID NO: 10; and human OPG is SEQ ID NO:
11. Conserved amino acids indicated with *.

| | | |
|---|---|---|
| muTNF-R2 | MAP-AALWVALVFELQLWATGHTVPAQ-VVLTPYK------PEPGYECQIS--QEYYD | 48 |
| huTNF-R2 | MAP-VAVWAALAVGLELWAAAHALPAQ-VAFTPYA------PEPGSTCRL---REYYD | 47 |
| HDTEA84 | MRALE-GPGLSLLCLVLALPALLPVPAVRGVAETPTY------PWR------------DA | 41 |
| huOPG | MNK------LLCCALVFLDISIKWTTQ-ETFPPKY------------------LHYDE | 33 |
| HSLJD37R. | MGTSPSSSTALASCSRIARRATATMIAGS-LLLLGFLSTTTAQPEQKASNLIGTYRHVDR | 59 |
| | | . |
| muTNF-R2 | RKAQMC-CAKCPPGQYVKHFCNKTSDTVCADCEASMYTQVWNQFRTCLSCSSSCTTDQVE | 107 |
| huTNF-R2 | QTAQMC-CSKCSPGQHAKVFCTKTSDTVCDSCEDSTYTQLWNWVPECLSCGSRCSSDQVE | 106 |
| HDTEA84 | ETGERLVCAQCPPGTFVQRPCRRDSPMTCGPCPPRHYTQFWNYLERCRYCNVLCGEREEE | 101 |
| huOPG | ETSHQLLCDKCPPGTYLKQHCTAKWKTVCAPCPDHYYTDSWHTSDECLYCSPVCKELQYV | 93 |
| HSLJD37R | ATGQVLTCDKCPAGTYVSEHCTNTSCASXSSCPVGTFTRHENGIEKCHDCSQPCPWPMIE | 119 |
| | * .* * * * .* . * * * | |
| muTNF-R2 | IRACTKQQNRVCACEAGRYCALKTHSGSCRQCMRLSKCGPGFGVASSRAPNGNVLCKACA | 167 |
| huTNF-R2 | TQACTREQNRICTCRPGWYCALSKQEG-CRLCAPLRKCRPGFGVARPGTETSDVVCKPCA | 165 |
| HDTEA84 | ARACHATHNRACRCRTGFF----AHAG---FCLEHASCPPGAGVIAPGTPSQNTQCQPCP | 154 |
| huOPG | KQECNRTHNRVCECKEGRY-----LEI--EFCLKHRSCPPGFGVVQAGTPERNTVCKRCP | 146 |
| HSLJD37R | KLPCAALTDRECTCPPGMF-----QSN--ATCAPHTVCPVGWGVRKKGTETEDVRCKQCA | 172 |
| | * * * * * . * * * ** . *. * | |
| muTNF-R2 | PGTFSDTTSSTDVCRPHRICSILAIPGNASTDAVCAPESPTLSAIPRTLYVSQPEPTRSQ | 227 |
| huTNF-R2 | PGTFSNTTSSTDICRPHQICNVVAIPGNASMDAVCTSTSPTRSMAPGAVHLPQPVSTRSQ | 225 |
| HDTEA84 | PGTFSASSSSSEQCQPHRNCTALGLALN----------------VPGS---SSHDTLCTS | 195 |
| huOPG | DGFFSNETSSKAPCRKHTNCSVFGLLLTQ----------------KGN---ATHDNICSG | 187 |
| HSLJD37R | RGYFSDVPSSVMX-AKH----------------------------------TQTVWIRT- | 196 |
| | * ** ** * * | |

TABLE 4

Rodent, e.g., mouse, 427152#4 RANK-like (RANKL; SEQ ID NO:
12 and 13).

| | |
|---|---|
| ggcacgaggg cgtttggcgc ggaagtgcta ccaagctgcg gaaagcgtga gtctggagca | 60 |
| cagcactggc gagtagcagg aataaacacg tttggtgaga gcc atg gca ctc aag | 115 |
| Met Ala Leu Lys | |
| gtc cta cct cta cac agg acg gtg ctc ttc gct gcc att ctc ttc cta | 163 |
| Val Leu Pro Leu His Arg Thr Val Leu Phe Ala Ala Ile Leu Phe Leu | |
| −25 −20 −15 −10 | |
| ctc cac ctg gca tgt aaa gtg agt tgc gaa acc gga gat tgc agg cag | 211 |
| Leu His Leu Ala Cys Lys Val Ser Cys Glu Thr Gly Asp Cys Arg Gln | |
| −5 −1 1 5 | |
| cag gaa ttc aag gat cga tct gga aac tgt gtc ctc tgc aaa cag tgc | 259 |
| Gln Glu Phe Lys Asp Arg Ser Gly Asn Cys Val Leu Cys Lys Gln Cys | |
| 10 15 20 | |
| gga cct ggc atg gag ttg tcc aag gaa tgt ggc ttc ggc tat ggg gag | 307 |
| Gly Pro Gly Met Glu Leu Ser Lys Glu Cys Gly Phe Gly Tyr Gly Glu | |
| 25 30 35 | |
| gat gca cag tgt gtg ccc tgc agg ccg cac cgg ttc aag gaa gac tgg | 355 |
| Asp Ala Gln Cys Val Pro Cys Arg Pro His Arg Phe Lys Glu Asp Trp | |
| 40 45 50 55 | |
| ggt ttc cag aag tgt aag cca tgt gcg gac tgt gcg ctg gtg aac cgc | 403 |
| Gly Phe Gln Lys Cys Lys Pro Cys Ala Asp Cys Ala Leu Val Asn Arg | |
| 60 65 70 | |
| ttt cag agg gcc aac tgc tca cac acc agt gat gct gtc tgc ggg gac | 451 |
| Phe Gln Arg Ala Asn Cys Ser His Thr Ser Asp Ala Val Cys Gly Asp | |
| 75 80 85 | |
| tgc ctg cca gga ttt tac cgg aag acc aaa ctg gtt ggt ttt caa gac | 499 |
| Cys Leu Pro Gly Phe Tyr Arg Lys Thr Lys Leu Val Gly Phe Gln Asp | |
| 90 95 100 | |
| atg gag tgt gtg ccc tgc gga gac cca cct cct ccc tac gaa cca cac | 547 |
| Met Glu Cys Val Pro Cys Gly Asp Pro Pro Pro Pro Tyr Glu Pro His | |
| 105 110 115 | |
| tgt gag tgatgtgcca agtggcagca gacctttaaa aaaaaaagaa aaaaaaacaa | 603 |
| Cys Glu | |
| 120 | |
| acaaaaacaa aaaaaaaaaa aaaaaaaaaa aaa | 636 |
| MALKVLPLHR TVLFAAILFL LHLACKVSCE TGDCRQQEFK DRSGNCVLCK QCGPGMELSK | |
| ECGFGYGEDA QCVPCRPHRF KEDWGFQKCK PCADCALVNR FQRANCSHTS DAVCGDCLPG | |
| FYRKTKLVGF QDMECVPCGD PPPPYEPHCE | |
| Primate, e.g., human, putative homolog of murine Rank-like (SEQ ID | |
| NO: 14 and 15). | |
| cgcgctgagg tggatttgta ccggagtccc atttgggagc aagagccatc tactcgtccg | 60 |
| ttaccggcct tcccacc atg gat tgc caa gaa aat gag tac tgg gac caa | 110 |
| Met Asp Cys Gln Glu Asn Glu Tyr Trp Asp Gln | |
| 1 5 10 | |
| tgg gga cgg tgt gtc acc tgc caa cgg tgt ggt cct gga cag gag cta | 158 |
| Trp Gly Arg Cys Val Thr Cys Gln Arg Cys Gly Pro Gly Gln Glu Leu | |
| 15 20 25 | |
| tcc aag gat tgt ggt tat gga gag ggt gga gat gcc tac tgc aca gcc | 206 |
| Ser Lys Asp Cys Gly Tyr Gly Glu Gly Gly Asp Ala Tyr Cys Thr Ala | |
| 30 35 40 | |
| tgc cct cct cgc agt aca aaa gca gct ggg gcc acc aca aat gtc aga | 254 |
| Cys Pro Pro Arg Ser Thr Lys Ala Ala Gly Ala Thr Thr Asn Val Arg | |
| 45 50 55 | |
| gtt gca tca cct gtg ctg tca tca atc gtg ttc aga agg ttc aac tgc | 302 |
| Val Ala Ser Pro Val Leu Ser Ser Ile Val Phe Arg Arg Phe Asn Cys | |
| 60 65 70 75 | |
| aca gtn acc tct nat gct gtc tgt ggg gga ngg ttt gcc caa gtt tct | 350 |
| Thr Xaa Thr Ser Xaa Ala Val Cys Gly Gly Xaa Phe Ala Gln Val Ser | |
| 80 85 90 | |
| aac cga aag aca cgc cat tgg aag gct gcc agg acc aag gat ggc atc | 398 |
| Asn Arg Lys Thr Arg His Trp Lys Ala Ala Arg Thr Lys Asp Gly Ile | |
| 95 100 105 | |
| ccg tgg cac aaa gnc aga ccc cca act tct gan ggt tnc aaa gtg nct | 446 |
| Pro Trp His Lys Xaa Arg Pro Pro Thr Ser Xaa Gly Xaa Lys Val Xaa | |
| 110 115 120 | |
| ttc caa ttg gag ctt aat ggg agg can a | 474 |
| Phe Gln Leu Glu Leu Asn Gly Arg Xaa | |
| 125 130 | |
| MDCQENEYWD QWGRCVTCQR CGPGQELSKD CGYGEGGDAY CTACPPRSTK AAGATTNVRV | |
| ASPVLSSIVF RRFNCTxTSx AVCGGxFAQV SNRKTRHWKA ARTKDGIPWH KxRPPTSxGx | |
| KVxFQLELNG Rx | |
| Additional primate, e.g., human, putative homologue of murine | |
| RANKL (SEQ ID NO: 16 and 17). | |
| cgcgctgagg tggatttgta ccggagtccc atttgggagc aagagccatc tactcgtccg | 60 |
| ttaccggcct tcccacc atg gat tgc caa gaa aat gag tac tgg gac caa | 110 |
| Met Asp Cys Gln Glu Asn Glu Tyr Trp Asp Gln | |
| 1 5 10 | |
| tgg gga cgg tgt gtc acc tgc caa cgg tgt ggt cct gga cag gag cta | 158 |
| Trp Gly Arg Cys Val Thr Cys Gln Arg Cys Gly Pro Gly Gln Glu Leu | |
| 15 20 25 | |
| tcc aag gat tgt ggt tat gga gag ggt gga gat gcc tac tgc aca gcc | 206 |
| Ser Lys Asp Cys Gly Tyr Gly Glu Gly Gly Asp Ala Tyr Cys Thr Ala | |
| 30 35 40 | |
| tgc cct cct cgc agg tac aaa agc agc tgg ggc cac cac aaa tgt cag | 254 |
| Cys Pro Pro Arg Arg Tyr Lys Ser Ser Trp Gly His His Lys Cys Gln | |
| 45 50 55 | |
| agt tgc atc acc tgt gct gtc atc aat cgt gtt cag aag gtc caa ctg | 302 |
| Ser Cys Ile Thr Cys Ala Val Ile Asn Arg Val Gln Lys Val Gln Leu | |
| 60 65 70 75 | |
| cac agc taacctctna tgctgtctgt ggggatgttt gncccaagtt ctnaccgaaa | 358 |
| His Ser | |
| agacacgcca tgggaaggct ggcaggacca ngaatggccn tcccgtggca gaaagccaga | 418 |
| ccccccaacn nctgnaggtt ccaatgtggc cttnccattt ggaagcttan tgggaaggca | 478 |
| gatgncaacc caaagtggcc ccttcaggga ggccaaaatt tgttggcaat gggtgnagca | 538 |
| gcntgcca | 546 |
| MDCQENEYWD QWGRCVTCQR CGPGQELSKD CGYGEGGDAY CTACPPRRYK SSWGHEKCQS | |
| CITCAVINRV QKVQLHS | |
| variant primate, e.g., human, sequence (SEQ ID NO: 18 and 19): | |
| cgcgctgagg tggatttgta ccggagtccc atttgggagc aagagccatc tactcgtccg | 60 |
| ttaccggcct tcccacc atg gat tgc caa gaa aat gag tac tgg gac caa | 110 |
| Met Asp Cys Gln Glu Asn Glu Tyr Trp Asp Gln | |
| 1 5 10 | |
| tgg gga cgg tgt gtc acc tgc caa cgg tgt ggt cct gga cag gag cta | 158 |
| Trp Gly Arg Cys Val Thr Cys Gln Arg Cys Gly Pro Gly Gln Glu Leu | |
| 15 20 25 | |
| tcc aag gat tgt ggt tat gga gag ggt gga gat gcc tac tgc aca gcc | 206 |
| Ser Lys Asp Cys Gly Tyr Gly Glu Gly Gly Asp Ala Tyr Cys Thr Ala | |
| 30 35 40 | |
| tgc cct cct cgc agg tac aaa agc agc tgg ggc cac cac aaa tgt cag | 254 |
| Cys Pro Pro Arg Arg Tyr Lys Ser Ser Trp Gly His His Lys Cys Gln | |
| 45 50 55 | |
| agt tgc atc acc tgt gct gtc atc aat cgt gtt cag aag gtc aac tgc | 302 |
| Ser Cys Ile Thr Cys Ala Val Ile Asn Arg Val Gln Lys Val Asn Cys | |
| 60 65 70 75 | |
| aca gct acc tct aat gct gtc tgt ggg gac tgt ttg ccc agg ttc tac | 350 |
| Thr Ala Thr Ser Asn Ala Val Cys Gly Asp Cys Leu Pro Arg Phe Tyr | |
| 80 85 90 | |
| cga aag aca cgc att gga ggc ctg cag gac caa gag tgc atc ccg tgc | 398 |
| Arg Lys Thr Arg Ile Gly Gly Leu Gln Asp Gln Glu Cys Ile Pro Cys | |
| 95 100 105 | |
| acg aag cag acc ccc acc tct gag gtt caa tgt gcc ttc cag ttg agc | 446 |
| Thr Lys Gln Thr Pro Thr Ser Glu Val Gln Cys Ala Phe Gln Leu Ser | |
| 110 115 120 | |
| tta gtg gag gca gat gca ccc aca gtg ccc cct cag gag gcc aca ctt | 494 |
| Leu Val Glu Ala Asp Ala Pro Thr Val Pro Pro Gln Glu Ala Thr Leu | |
| 125 130 135 | |
| gtt gca ctg gtg agc agc ctg cta gtg gtg ttt acc ctg gcc ttc ctg | 542 |
| Val Ala Leu Val Ser Ser Leu Leu Val Val Phe Thr Leu Ala Phe Leu | |
| 140 145 150 155 | |
| ggg ctc ttc ttc ctc tac tgc aag cag ttc ttc aac aga cat tgc cag | 590 |
| Gly Leu Phe Phe Leu Tyr Cys Lys Gln Phe Phe Asn Arg His Cys Gln | |
| 160 165 170 | |
| cgt gga ggt ttg ctg cag ttt gag gct gat aaa aca gca aag gag gaa | 638 |
| Arg Gly Gly Leu Leu Gln Phe Glu Ala Asp Lys Thr Ala Lys Glu Glu | |
| 175 180 185 | |
| tct ctc ttc ccc gtg cca ccc agc aag gag acc agt gct gag tcc caa | 686 |
| Ser Leu Phe Pro Val Pro Pro Ser Lys Glu Thr Ser Ala Glu Ser Gln | |
| 190 195 200 | |
| gtc tct tgg gcc cct ggc agc ctt gcc cag ttg ttc tct ctg gac tct | 734 |
| Val Ser Trp Ala Pro Gly Ser Leu Ala Gln Leu Phe Ser Leu Asp Ser | |
| 205 210 215 | |
| gtt cct ata cca caa cag cag cag ggg cct gaa atg tgatgtccac | 780 |
| Val Pro Ile Pro Gln Gln Gln Gln Gly Pro Glu Met | |
| 220 225 230 | |
| angagctaat accctacaga tggggcatat cctatcccat cccaccagag gattgattct | 840 |
| ccatttcaca aggactgatc tggagcattt cttgcttccc tgttgtagtc tggggagcca | 900 |
| gattccacat tcatgggact accagacatg tt | 932 |
| MDCQENEYWD QWGRCVTCQR CGPGQELSKD CGYGEGGDAY CTACPPRRYK SSWGHHKCQS | |
| CITCAVINRV QKVNCTATSN AVCGDCLPRF YRKTRIGGLQ DQECIPCTKQ TPTSEVQCAF | |
| QLSLVEADAP TVPPQEATLV ALVSSLLWF TLAFLGLFFL YCKQFFNRHC QRGGLLQFEA | |
| DKTAKEESLF PVPPSKETSA ESQVSWAPGS LAQLFSLDSV PIPQQQQGPE M | |
| alignment of mouse and human RANKL (residue numbering different | |
| from above): | |

| | | |
|---|---|---|
| mRANKL | 1 MALKVLPLHRTVLFAAILFLLHLACKVSCETGDCRQQEFKDRSGNCVLCK | 50 |
| hRANKL | 1 MDCQENEYWDQWGRCVTCQ | 19 |
| | **...*. *. * ** *. | |
| mRANKL | 51 QCGPGMELSKECGFGYGEDAQCVPCRPHRFKEDWGFQKCKPCADCALVNR | 100 |
| hRANKL | 20 RCGPGQELSKDCGYGEGGDAYCTACPPRRYKSSWGHHKCQSCITCAVINR | 69 |
| | .**** ****.**.* * ** * * *.*.* ** .**. * **..** | |
| mRANKL | 101 FQRANCSHTSDAVCGDCLPGFYRKTKLVGFQDMECVPCG----------- | 139 |
| hRANKL | 70 VQKVNCTATSNAVCGDCLPRFYRKTRIGGLQDQECIPCTKQTPTSEVQCA | 119 |
| | *. **. ** ******** *****.. * ** **.** | |
| mRANKL | 140 --------DPP--PP-------------------------------YEPH | 148 |
| hRANKL | 120 FQLSLVEADAPTVPPQEATLVALVSSLLVVFTLAFLGLFFLYCKQFFNRH | 169 |
| | * * ** . * | |
| mRANKL | 149 CE | 150 |
| hRANKL | 170 CQRGGLLQFEADKTAKEESLFPVPPSKETSAESQVSWAPGSLAQLFSLDS | 219 |
| | *. | |
| mRANKL | 151 | 151 |
| hRANKL | 220 VPIPQQQQGPEM | 231 |

Interesting features of the HDTEA84 (SEQ ID NO: 2) include: predicted signal sequence from about −11 to −1; TNF receptor Cys rich domains I (about glu21-pro61), II (about cys62-cys102), III (about arg103-cysl39), and IV (about g1n140-cys182); and unique region from about thr183-his289. Features for the HSLJD37R (SEQ ID NO: 5 form), partly based on alignment with HDTEA84: signal sequence from about −41 to −1; TNF receptor Cys rich domains I (about g1n1-ser49), II (about cys50-cys90), III (about thr91-cys127), and IV (about lys128-cys170); and transmembrane segment from about ile313-ile329. Similar alignment of the other variants will identify similar features. Segments including combinations or excluding such segments may be desired.

Interesting features of the rodent RANKL (SEQ ID NO: 13) include: signal sequence from about −29 to −1; TNF receptor Cys rich domain I (about asp4-pro45), II (about cys46-cys85), and III (about gly86-cys106). Interesting features of the primate RANKL (SEQ ID NO: 19) include: TNF receptor Cys rich domain I (about met1-ala43), II (about cys44-cys83), and III (about gly84-cys104); transmembrane segment from about leu139-leu155. Alignment with other TNF receptors will identify additional interesting corresponding features. Segments with boundaries at these positions may be especially interesting.

Hybridization signals with RANKL were detected with rodent, e.g., mouse sequence, in CH12 (B cell line), rag-1 thymus, rag-1 heart, rag-1 brain (strongest signal), rag-1 testes, rag-1 liver, normal lung, rag-1 lung, asthmatic lung, tolerized and challenged lung, Nippo-infected lung, Nippo IL-4 K.O. lung, Nippo anti-IL-5 lung, influenza lung, guinea pig allergic lung, w.t. stomach, and w.t. colon on a 3 day exposure at −80° C. with a screen. On a 2 week exposure at −80° C. with screen, signals were also detected in the following libraries: Mel 14+ naive, Mel14+ Th1, Mel14+ Th2, Th1 3 week Bl/6, large B cell, bEnd3+TNFα+IL-10, guinea pig normal lung, and Rag Hh- colon. Probes of human libraries with rodent sequence provided: detectable signals in monkey asthma lung 4 h (1.6-2.0 kb) and adult placenta (2.5-3.0 kb) on a 3 day exposure at −80° C with screen. On a 2 week exposure at −80° C. with screen, signals were also detected in the following libraries: CD1a+95% DC activated, CHA (kidney epithelial carcinoma cell line), monkey lung normal, psoriasis skin, fetal lung, fetal ovary, fetal testes, and fetal spleen.

The structural homology of HDTEA84, HSLJD37R, and RANKL to members of the TNF receptor family suggests related function of these molecules. See, e.g., Lacey, et al. (1998) Cell 93:165-176. The sequences, however, both lack a transmembrane segment, suggesting that the proteins are soluble receptor forms. They may well also have membrane bound forms resulting, e.g., from alternatively spliced transcript variants. The soluble forms are likely to be antagonists of the ligand, e.g., blocking the binding of ligand to a membrane bound form of signaling receptor. Thus, these molecules may be useful in the treatment of abnormal immune or developmental disorders.

The natural antigens should be capable of modulating various biochemical responses which lead to biological or physiological responses in target cells. The embodiments characterized herein are from primate, e.g., human, but other species variants almost surely exist, e.g., rodents, etc. See below. The descriptions below are directed, for exemplary purposes, to primate HDTEA84, HSLJD37R, or RANKL, but are likewise applicable to related embodiments from other species.

The HDTEA84, HSLJD37R, and RANKL clones were assembled through the careful analysis of ESTs present in various databases, e.g., Merck-WashU public database. These genes exhibit structural motifs characteristic of a member of the TNF receptor family. Compare, e.g., with the TNF receptor, NGF-receptor, and FAS receptor. Table 1 illustrates the nucleic acid and predicted amino acid sequences for primate, e.g., human, HDTEA84. The ESTs were identified from several different libraries.

Table 2 illustrates partial nucleic acid and predicted amino acid sequences for primate, e.g., human, HSLJD37R. The ESTs were identified from several different libraries derived from: smooth muscle, pancreas tumor, adipocytes, HUVEC cells, adult pulmonary, endothelial cells, prostate cell line PC3, microvascular endothelial cells, fetal heart, and dendritic cells. Other sequences were detected in libraries from: multiple sclerosis lesions, breast, kidney, and germinal center B cells.

Table 4 gives sequence of various mammalian genes designated RANKL.

The structural homology of these genes to the TNF ligand family suggests related function of these molecules. Receptor family antagonists, or agonists, may act as a co-stimulatory molecule for regulation of T cell mediated cell activation, and may in fact, cause a shift of T helper cell types, e.g., between Th1 and Th2. Alternatively, the ligands for the receptors may serve to regulate cell proliferation or development.

TNF ligand molecules typically modulate cell proliferation, viability, and differentiation. For example, TNF and FAS can kill cells expressing their respective receptors, including fibroblasts, liver cells, and lymphocytes. Some members of this class of ligands exhibit effects on cellular proliferation of cells expressing their respective receptors, e.g., B cells expressing CD40. These effects on proliferation may also effect subsequent differentiation steps, and may lead, directly or indirectly, to changes in cytokine expression profiles.

The members of the TNF ligand family also exhibit costimulation effects, which may also regulate cellular differentiation or apoptosis. Receptor expressing cells may be protected from activation induced cell death (AICD) or apoptosis. For example, CD40 ligand can have effects on T and B lymphocytes.

The embodiments characterized herein are from human, but additional sequences for proteins in other mammalian species, e.g., primates and rodents, will also be available. See below. The descriptions below are directed, for exemplary purposes, to a human HDTEA84, HSLJD37R, or RANKL, but are likewise applicable to related embodiments from other species.

II. Purified Receptor

Human HDTEA84 amino acid sequence is shown in SEQ ID NO: 2; primate HSLJD37R amino acid sequences are shown in SEQ ID NO: 4, 6, and 8; murine RANKL sequence is shown in SEQ ID NO: 13, and three primate forms of RANKL sequence are shown in SEQ ID NO: 15, 17, and 19. These amino acid sequences, provided amino to carboxy, are important in providing sequence information in the antigen allowing for distinguishing the protein from other proteins and exemplifying numerous variants. Moreover, the peptide sequences allow preparation of peptides to generate antibodies to recognize such segments, and nucleotide sequences allow preparation of oligonucleotide probes, both of which are strategies for detection or isolation, e.g., cloning, of genes or cDNAs encoding such sequences.

As used herein, the term “human HDTEA84” shall encompass, when used in a protein context, a protein having amino acid sequence shown in SEQ ID NO: 2. Significant fragments of such a protein should preserve at least some of the properties of the full length protein. Other essentially identical proteins may be found in other primates. In addition, binding components, e.g., antibodies, typically bind to an HDTEA84 with high affinity, e.g., at least about 100 nM, usually better than about 30 nM, preferably better than about 10 nM, and more preferably at better than about 3 nM. Homologous proteins would be found in mammalian species other than human, e.g., primates or rodents. Non-mammalian species should also possess structurally or functionally related genes and proteins, e.g., birds or amphibians. A similar term applies to HSLJD37R or RANKL.

The term “polypeptide” as used herein includes a significant fragment or segment, and encompasses a stretch of amino acid residues of at least about 8 amino acids, generally at least about 12 amino acids, typically at least about 16 amino acids, preferably at least about 20 amino acids, and, in particularly preferred embodiments, at least about 30 or more amino acids, e.g., 35, 40, 45, 50, 70, 90, and more. In certain embodiments, there will be a plurality of distinct, e.g., nonoverlapping, segments of the specified length. Typically, the plurality will be at least two, more usually at least three, and preferably 5, 7, or even more. While the length minima are provided, longer lengths, of various sizes, may be appropriate, e.g., one of length 7, and two of length 12.

The term “binding composition” refers to molecules that bind with specificity to the respective receptor, e.g., HDTEA84, e.g., in a cell adhesion pairing type fashion, or an antibody-antigen interaction. Other compounds include, e.g., proteins, which specifically associate with HDTEA84, including in a natural physiologically relevant protein-protein interaction, either covalent or non-covalent. The molecule may be a polymer, or chemical reagent. A functional analog may be an antigen with structural modifications, or it may be a molecule which has a molecular shape which interacts with the appropriate binding determinants. The compounds may serve as agonists or antagonists of the binding interaction, see, e.g., Goodman, et al. (eds. 1990) Goodman & Gilman's: The Pharmacological Bases of Therapeutics (8th ed.) Pergamon Press.

Substantially pure typically means that the protein is free from other contaminating proteins, nucleic acids, or other biologicals derived from the original source organism. Purity may be assayed by standard methods, typically by weight, and will ordinarily be at least about 40% pure, generally at least about 50% pure, often at least about 60% pure, typically at least about 80% pure, preferably at least about 90% pure, and in most preferred embodiments, at least about 95% pure. Carriers or excipients will often be added.

Solubility of a polypeptide or fragment depends upon the environment and the polypeptide. Many parameters affect polypeptide solubility, including temperature, electrolyte environment, size and molecular characteristics of the polypeptide, and nature of the solvent. Typically, the temperature at which the polypeptide is used ranges from about 4° C. to about 65° C. Usually the temperature at use is greater than about 18° C. For diagnostic purposes, the temperature will usually be about room temperature or warmer, but less than the denaturation temperature of components in the assay. For therapeutic purposes, the temperature will usually be body temperature, typically about 37° C. for humans and mice, though under certain situations the temperature may be raised or lowered in situ or in vitro.

The size and structure of the polypeptide should generally be in a substantially stable state, and usually not in a denatured state. The polypeptide may be associated with other polypeptides in a quaternary structure, e.g., to confer solubility, or associated with lipids or detergents in a manner which approximates natural lipid bilayer interactions.

The solvent and electrolytes will usually be a biologically compatible buffer, of a type used for preservation of biological activities, and will usually approximate a physiological aqueous solvent. Usually the solvent will have a neutral pH, typically between about 5 and 10, and preferably about 7.5. On some occasions, one or more detergents will be added, typically a mild non-denaturing one, e.g., CHS (cholesteryl hemisuccinate) or CHAPS (3-[3-cholamidopropyl)dimethylammonio]-1-propane sulfonate), or a low enough concentration as to avoid significant disruption of structural or physiological properties of the protein.

III. Physical Variants

This invention also encompasses proteins or peptides having substantial amino acid sequence identity with the amino acid sequence of the receptors, e.g., HDTEA84. The variants include species, polymorphic, or allelic variants.

Amino acid sequence homology, or sequence identity, is determined by optimizing residue matches, if necessary, by introducing gaps as required. See also Needleham, et al. (1970) J. Mol. Biol. 48:443-453; Sankoff, et al. (1983) Chapter One in Time Wars, String Edits, and Macromolecules: The Theory and Practice of Sequence Comparison, Addison-Wesley, Reading, Mass.; and software packages from IntelliGenetics, Mountain View, Calif.; and the University of Wisconsin Genetics Computer Group, Madison, Wis. Sequence identity changes when considering conservative substitutions as matches. Conservative substitutions typically include substitutions within the following groups: glycine, alanine; valine, isoleucine, leucine; aspartic acid, glutamic acid; asparagine, glutamine; serine, threonine; lysine, arginine; and phenylalanine, tyrosine. Homologous amino acid sequences are typically intended to include natural polymorphic or allelic and interspecies variations in each respective protein sequence. Typical homologous proteins or peptides will have from 25-100% identity (if gaps can be introduced), to 50-100% identity (if conservative substitutions are included) with the amino acid sequence of the HDTEA84. Identity measures will be at least about 35%, generally at least about 40%, often at least about 50%, typically at least about 60%, usually at least about 70%, preferably at least about 80%, and more preferably at least about 90%.

For sequence comparison, typically one sequence acts as a reference sequence, to which test sequences are compared. When using a sequence comparison algorithm, test and reference sequences are input into a computer, subsequence coordinates are designated, if necessary, and sequence algorithm program parameters are designated. The sequence comparison algorithm then calculates the percent sequence identity for the test sequence(s) relative to the reference sequence, based on the designated program parameters.

Optical alignment of sequences for comparison can be conducted, e.g., by the local homology algorithm of Smith and Waterman (1981) Adv. Appl. Math. 2:482, by the homology alignment algorithm of Needlman and Wunsch (1970) J. Mol. Biol. 48:443, by the search for similarity method of Pearson and Lipman (1988) Proc. Nat'l Acad. Sci. USA 85:2444, by computerized implementations of these algorithms (GAP, BESTFIT, FASTA, and TFASTA in the Wisconsin Genetics Software Package, Genetics Computer Group, 575 Science Dr., Madison, Wis.), or by visual inspection (see generally Ausubel et al., supra).

One example of a useful algorithm is PILEUP. PILEUP creates a multiple sequence alignment from a group of related sequences using progressive, pairwise alignments to show relationship and percent sequence identity. It also plots a tree or dendrogram showing the clustering relationships used to create the alignment. PILEUP uses a simplification of the progressive alignment method of Feng and Doolittle (1987) J. Mol. Evol. 35:351-360. The method used is similar to the method described by Higgins and Sharp (1989) CABIOS 5:151-153. The program can align up to 300 sequences, each of a maximum length of 5,000 nucleotides or amino acids. The multiple alignment procedure begins with the pairwise alignment of the two most similar sequences, producing a cluster of two aligned sequences. This cluster is then aligned to the next most related sequence or cluster of aligned sequences. Two clusters of sequences are aligned by a simple extension of the pairwise alignment of two individual sequences. The final alignment is achieved by a series of progressive, pairwise alignments. The program is run by designating specific sequences and their amino acid or nucleotide coordinates for regions of sequence comparison and by designating the program parameters. For example, a reference sequence can be compared to other test sequences to determine the percent sequence identity relationship using the following parameters: default gap weight (3.00), default gap length weight (0.10), and weighted end gaps.

Another example of algorithm that is suitable for determining percent sequence identity and sequence similarity is the BLAST algorithm, which is described Altschul, et al. (1990) J. Mol. Biol. 215:403-410. Software for performing BLAST analyses is publicly available through the National Center for Biotechnology Information (http:www.ncbi.nlm.nih.gov/). This algorithm involves first identifying high scoring sequence pairs (HSPs) by identifying short words of length W in the query sequence, which either match or satisfy some positive-valued threshold score T when aligned with a word of the same length in a database sequence. T is referred to as the neighborhood word score threshold (Altschul, et al., supra). These initial neighborhood word hits act as seeds for initiating searches to find longer HSPs containing them. The word hits are then extended in both directions along each sequence for as far as the cumulative alignment score can be increased. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLAST program uses as defaults a wordlength (W) of 11, the BLOSUM62 scoring matrix (see Henikoff and Henikoff (1989) Proc. Nat'l Acad. Sci. USA 89:10915) alignments (B) of 50, expectation (E) of 10, M=5, N=4, and a comparison of both strands.

In addition to calculating percent sequence identity, the BLAST algorithm also performs a statistical analysis of the similarity between two sequences (see, e.g., Karlin and Altschul (1993) Proc. Nat'l Acad. Sci. USA 90:5873-5787). One measure of similarity provided by the BLAST algorithm is the smallest sum probability (P(N)), which provides an indication of the probability by which a match between two nucleotide or amino acid sequences would occur by chance. For example, a nucleic acid is considered similar to a reference sequence if the smallest sum probability in a comparison of the test nucleic acid to the reference nucleic acid is less than about 0.1, more preferably less than about 0.01, and most preferably less than about 0.001.

A further indication that two nucleic acid sequences of polypeptides are substantially identical is that the polypeptide encoded by the first nucleic acid is immunologically cross reactive with the polypeptide encoded by the second nucleic acid, as described below. Thus, a polypeptide is typically substantially identical to a second polypeptide, for example, where the two peptides differ only by conservative substitutions. Another indication that two nucleic acid sequences are substantially identical is that the two molecules hybridize to each other under stringent conditions, as described below.

The isolated HDTEA84, HSLJD37R, or RANKL DNA can be readily modified by nucleotide substitutions, nucleotide deletions, nucleotide insertions, and inversions of nucleotide stretches. These modifications result in novel DNA sequences which encode these antigens, their derivatives, or proteins having similar physiological, immunogenic, antigenic, or other functional activity. These modified sequences can be used to produce mutant antigens or to enhance expression. Enhanced expression may involve gene amplification, increased transcription, increased translation, and other mechanisms. “Mutant HDTEA84” encompasses a polypeptide otherwise falling within the sequence identity definition of the HDTEA84 as set forth above, but having an amino acid sequence which differs from that of HDTEA84 as normally found in nature, whether by way of deletion, substitution, or insertion. This generally includes proteins having significant identity with a protein having sequence of SEQ ID NO: 2, and as sharing various biological activities, e.g., antigenic or immunogenic, with those sequences, and in preferred embodiments contain most of the full length disclosed sequences. Full length sequences will typically be preferred, though truncated versions, e.g., soluble constructs and intact domains, will also be useful, likewise, genes or proteins found from natural sources are typically most desired. Similar concepts apply to different HDTEA84 proteins, particularly those found in various warm blooded animals, e.g., mammals and birds. These descriptions are generally meant to encompass all HDTEA84 proteins, not limited to the particular human embodiment specifically discussed. Similar concepts apply to the HSLJD37R.

HDTEA84, HSLJD37R, or RANKL mutagenesis can also be conducted by making amino acid insertions or deletions. Substitutions, deletions, insertions, or any combinations may be generated to arrive at a final construct. Insertions include amino- or carboxy- terminal fusions. Random mutagenesis can be conducted at a target codon and the expressed mutants can then be screened for the desired activity. Methods for making substitution mutations at predetermined sites in DNA having a known sequence are well known in the art, e.g., by M13 primer mutagenesis or polymerase chain reaction (PCR) techniques. See, e.g., Sambrook, et al. (1989); Ausubel, et al. (1987 and Supplements); and Kunkel, et al. (1987) Methods in Enzymol. 154:367-382.

The present invention also provides recombinant proteins, e.g., heterologous fusion proteins using segments from these proteins. A heterologous fusion protein is a fusion of proteins or segments which are naturally not normally fused in the same manner. A similar concept applies to heterologous nucleic acid sequences. Fusion proteins will be useful as sources for cleaving, separating, and purifying portions thereof.

In addition, new constructs may be made from combining similar functional domains from other proteins. For example, target-binding or other segments may be “swapped” between different new fusion polypeptides or fragments. See, e.g., Cunningham, et al. (1989) Science 243:1330-1336; and O'Dowd, et al. (1988) J. Biol. Chem. 263:15985-15992.

The phosphoramidite method described by Beaucage and Carruthers (1981) Tetra. Letts. 22:1859-1862, will produce suitable synthetic DNA fragments. A double stranded fragment will often be obtained either by synthesizing the complementary strand and annealing the strand together under appropriate conditions or by adding the complementary strand using DNA polymerase with an appropriate primer sequence, e.g., PCR techniques.

IV. Functional Variants

The blocking of physiological response with HDTEA84, HSLJD37R, or RANKL may result from the inhibition of binding of the respective ligand to signaling form of receptor, e.g., transmembrane form of receptor, likely through competitive inhibition. Thus, in vitro assays of the present invention will often use isolated protein, soluble fragments comprising ligand binding segments of these proteins, or forms attached to solid phase substrates. These assays will also allow for the diagnostic determination of the effects of either binding segment mutations and modifications, or antigen mutations and modifications, e.g., HDTEA84, HSLJD37R, or RANKL analogs.

This invention also contemplates the use of competitive drug screening assays, e.g., where neutralizing antibodies to antigen or binding fragments compete with a test compound for binding to the protein, e.g., of natural protein sequence.

“Derivatives” of receptor antigens include amino acid sequence mutants from naturally occurring forms, glycosylation variants, and covalent or aggregate conjugates with other chemical moieties. Covalent derivatives can be prepared by linkage of functionalities to groups which are found in receptor amino acid side chains or at the N- or C- termini, e.g., by standard means. See, e.g., Lundblad and Noyes (1988) Chemical Reagents for Protein Modification, vols. 1-2, CRC Press, Inc., Boca Raton, Fla.; Hugli (ed. 1989) Techniques in Protein Chemistry, Academic Press, San Diego, Calif.; and Wong (1991) Chemistry of Protein Conjugation and Cross Linking, CRC Press, Boca Raton, Fla.

In particular, glycosylation alterations are included, e.g., made by modifying the glycosylation patterns of a polypeptide during its synthesis and processing, or in further processing steps. See, e.g., Elbein (1987) Ann. Rev. Biochem. 56:497-534. Also embraced are versions of the peptides with the same primary amino acid sequence which have other minor modifications, including phosphorylated amino acid residues, e.g., phosphotyrosine, phosphoserine, or phosphothreonine.

Fusion polypeptides between HDTEA84, HSLJD37R, or RANKL and other homologous or heterologous proteins are also provided. Many cytokine receptors or other surface proteins are multimeric, e.g., homodimeric entities, and a repeat construct may have various advantages, including lessened susceptibility to proteolytic cleavage. Typical examples are fusions of a reporter polypeptide, e.g., luciferase, with a segment or domain of a protein, e.g., a receptor-binding segment, so that the presence or location of the fused ligand may be easily determined. See, e.g., Dull, et al., U.S. Pat. No. 4,859,609. Other gene fusion partners include bacterial β-galactosidase, trpE, Protein A, β-lactamase, alpha amylase, alcohol dehydrogenase, yeast alpha mating factor, and detection or purification tags such as a FLAG sequence of His6 sequence. See, e.g., Godowski, et al. (1988) Science 241:812-816. Of particular interest are fusion constructs of the receptor with a membrane attachment domain.

Fusion peptides will typically be made by either recombinant nucleic acid methods or by synthetic polypeptide methods. Techniques for nucleic acid manipulation and expression are described generally, e.g., in Sambrook, et al. (1989) Molecular Cloning: A Laboratory Manual (2d ed.), vols. 1-3, Cold Spring Harbor Laboratory; and Ausubel, et al. (eds. 1993) Current Protocols in Molecular Biology, Greene and Wiley, NY. Techniques for synthesis of polypeptides are described, e.g., in Merrifield (1963) J. Amer. Chem. Soc. 85:2149-2156; Merrifield (1986) Science 232: 341-347; Atherton, et al. (1989) Solid Phase Peptide Synthesis: A Practical Approach, IRL Press, Oxford; and Grant (1992) Synthetic Peptides: A User's Guide, W. H. Freeman, NY.

This invention also contemplates the use of derivatives of HDTEA84, HSLJD37R, or RANKL other than variations in amino acid sequence or glycosylation. Such derivatives may involve covalent or aggregative association with chemical moieties. Covalent or aggregative derivatives will be useful as immunogens, as reagents in immunoassays, or in purification methods such as for affinity purification of binding partners, e.g., other antigens. An HDTEA84, HSLJD37R, or RANKL can be immobilized by covalent bonding to a solid support such as cyanogen bromide-activated SEPHAROSE, by methods which are well known in the art, or adsorbed onto polyolefin surfaces, with or without glutaraldehyde cross-linking, for use in the assay or purification of antibodies or an alternative binding composition. The HDTEA84, HSLJD37R, or RANKL can also be labeled with a detectable group, e.g., for use in diagnostic assays. Purification of receptor may be effected by an immobilized antibody or complementary binding partner.

A solubilized receptor or fragment of this invention can be used as an immunogen for the production of antisera or antibodies specific for binding to the antigen or fragments thereof. Purified antigen can be used to screen monoclonal antibodies or antigen-binding fragments, encompassing antigen binding fragments of natural antibodies, e.g., Fab, Fab′, F(ab)2, etc. Purified HDTEA84, HSLJD37R, or RANKL can also be used as a reagent to detect antibodies generated in response to the presence of elevated levels of the antigen or cell fragments containing the antigen, both of which may be diagnostic of an abnormal or specific physiological or disease condition. This invention contemplates antibodies raised against amino acid sequences encoded by nucleotide sequence shown in SEQ ID NO: 1, or 3, 5, or 7; or 12, 14, 16, or 18, or fragments of proteins containing it. In particular, this invention contemplates antibodies having binding affinity to or being raised against specific fragments which are predicted to lie outside of the lipid bilayer, both extracellular or intracellular.

The present invention contemplates the isolation of additional closely related species variants. Southern and Northern blot analysis should establish that similar genetic entities exist in other mammals. It is likely that these receptors are widespread in species variants, e.g., rodents, lagomorphs, carnivores, artiodactyla, perissodactyla, and primates.

The invention also provides means to isolate a group of related antigens displaying both distinctness and similarities in structure, expression, and function. Elucidation of many of the physiological effects of the molecules will be greatly accelerated by the isolation and characterization of additional distinct species variants of them. In particular, the present invention provides useful probes for identifying additional homologous genetic entities in different species.

The isolated genes will allow transformation of cells lacking expression of a corresponding receptor, e.g., either species types or cells which lack corresponding antigens and exhibit negative background activity. This should allow analysis of the function of receptor in comparison to untransformed control cells.

Dissection of critical structural elements which effect the various activation or differentiation functions mediated through these antigens is possible using standard techniques of modern molecular biology, particularly in comparing members of the related class. See, e.g., the homolog-scanning mutagenesis technique described in Cunningham, et al. (1989) Science 243:1339-1336; and approaches used in O'Dowd, et al. (1988) J. Biol. Chem. 263:15985-15992; and Lechleiter, et al. (1990) EMBO J. 9:4381-4390.

Intracellular functions would probably involve segments of the antigen which are normally accessible to the cytosol of transmembrane forms of the receptors. However, protein internalization may occur under certain circumstances, and interaction between intracellular components and “extracellular” segments may occur. The specific segments of interaction of receptor with other intracellular components may be identified by mutagenesis or direct biochemical means, e.g., cross-linking or affinity methods. Structural analysis by crystallographic or other physical methods will also be applicable. Further investigation of the mechanism of signal transduction will include study of associated components which may be isolatable by affinity methods or by genetic means, e.g., complementation analysis of mutants.

Further study of the expression and control of HDTEA84, HSLJD37R, or RANKL will be pursued. The controlling elements associated with the antigens should exhibit differential physiological, developmental, tissue specific, or other expression patterns. Upstream or downstream genetic regions, e.g., control elements, are of interest. In particular, physiological or developmental variants, e.g., multiple alternatively processed forms of the antigen might be found. See, e.g., SEQ ID NO: 2, 4, 6, 8, 13, 15, 17, or 19. Thus, differential splicing of message may lead to an assortment of membrane bound forms, soluble forms, and modified versions of antigen. See SEQ ID NO: 8 and 19.

Structural studies of the antigens will lead to design of new antigens, particularly analogs exhibiting agonist or antagonist properties on the molecule. This can be combined with previously described screening methods to isolate antigens exhibiting desired spectra of activities.

V. Antibodies

Antibodies can be raised to various receptors, including species, polymorphic, or allelic variants, and fragments thereof, both in their naturally occurring forms and in their recombinant forms. Additionally, antibodies can be raised to HDTEA84, HSLJD37R, or RANKL in either their active forms or in their inactive forms, including native or denatured versions. Anti-idiotypic antibodies are also contemplated.

Antibodies, including binding fragments and single chain versions, against predetermined fragments of the antigens can be raised by immunization of animals with conjugates of the fragments with immunogenic proteins. Monoclonal antibodies are prepared from cells secreting the desired antibody. These antibodies can be screened for binding to normal or defective HDTEA84, HSLJD37R, or RANKL, or screened for agdnistic or antagonistic activity, e.g., mediated through the antigen or its binding partner. Antibodies may be agonistic or antagonistic, e.g., by sterically blocking ligand binding. These monoclonal antibodies will usually bind with at least a K_{D} of about 1 mM, more usually at least about 300 μM, typically at least about 100 μM, more typically at least about 30 μM, preferably at least about 10 μM, and more preferably at least about 3 μM or better.

The antibodies of this invention can also be useful in diagnostic applications. As capture or non-neutralizing antibodies, they can be screened for ability to bind to the antigens without inhibiting binding by a partner. As neutralizing antibodies, they can be useful in competitive binding assays. They will also be useful in detecting or quantifying HDTEA84, HSLJD37R, or-RANKL protein or its binding partners. See, e.g., Chan (ed. 1987) Immunology: A Practical Guide, Academic Press, Orlando, Fla.; Price and Newman (eds. 1991) Principles and Practice of Immunoassay, Stockton Press, N.Y.; and Ngo (ed. 1988) Nonisotopic Immunoassay, Plenum Press, N.Y. Cross absorptions or other tests will identify antibodies which exhibit various spectra of specificities, e.g., unique or shared species specificities.

Further, the antibodies, including antigen binding fragments, of this invention can be potent antagonists that bind to the antigen and inhibit functional binding or inhibit the ability of a binding partner to elicit a biological response. They also can be useful as non-neutralizing antibodies and can be coupled to toxins or radionuclides so that when the antibody binds to antigen, a cell expressing it, e.g., on its surface, is killed. Further, these antibodies can be conjugated to drugs or other therapeutic agents, either directly or indirectly by means of a linker, and may effect drug targeting.

Antigen fragments may be joined to other materials, particularly polypeptides, as fused or covalently joined polypeptides to be used as immunogens. An antigen and its fragments may be fused or covalently linked to a variety of immunogens, such as keyhole limpet hemocyanin, bovine serum albumin, tetanus toxoid, etc. See Microbiology, Hoeber Medical Division, Harper and Row, 1969; Landsteiner (1962) Specificity of Serological Reactions, Dover Publications, New York; Williams, et al. (1967) Methods in Immunology and Immunochemistry, vol. 1, Academic Press, New York; and Harlow and Lane (1988) Antibodies: A Laboratory Manual, CSH Press, NY, for descriptions of methods of preparing polyclonal antisera.

In some instances, it is desirable to prepare monoclonal antibodies from various mammalian hosts, such as mice, rodents, primates, humans, etc. Description of techniques for preparing such monoclonal antibodies may be found in, e.g., Stites, et al. (eds.) Basic and Clinical Immunology (4th ed.), Lange Medical Publications, Los Altos, Calif., and references cited therein; Harlow and Lane (1988) Antibodies: A Laboratory Manual, CSH Press; Goding (1986) Monoclonal Antibodies: Principles and Practice (2d ed.), Academic Press, New York; and particularly in Kohler and Milstein (1975) in Nature 256:495-497, which discusses one method of generating monoclonal antibodies.

Other suitable techniques involve in vitro exposure of lymphocytes to the antigenic polypeptides or alternatively to selection of libraries of antibodies in phage or similar vectors. See, Huse, et al. (1989) “Generation of a Large Combinatorial Library of the Immunoglobulin Repertoire in Phage Lambda,” Science 246:1275-1281; and Ward, et al. (1989) Nature 341:544-546. The polypeptides and antibodies of the present invention may be used with or without modification, including chimeric or humanized antibodies. Frequently, the polypeptides and antibodies will be labeled by joining, either covalently or non-covalently, a substance which provides for a detectable signal. A wide variety of labels and conjugation techniques are known and are reported extensively in both the scientific and patent literature. Suitable labels include radionuclides, enzymes, substrates, cofactors, inhibitors, fluorescent moieties, chemiluminescent moieties, magnetic particles, and the like. Patents, teaching the use of such labels include U.S. Pat. Nos. 3,817,837; 3,850,752; 3,939,350; 3,996,345; 4,277,437; 4,275,149; and 4,366,241. Also, recombinant immunoglobulins may be produced, see Cabilly, U.S. Pat. No. 4,816,567; Moore, et al., U.S. Pat. No. 4,642,334; and Queen, et al. (1989) Proc. Nat'l Acad. Sci. USA 86:10029-10033.

The antibodies of this invention can also be used for affinity chromatography in isolating the protein. Columns can be prepared where the antibodies are linked to a solid support. See, e.g., Wilchek et al. (1984) Meth. Enzymol. 104:3-55.

Antibodies raised against each HDTEA84, HSLJD37R, or RANKL will also be useful to raise anti-idiotypic antibodies. These will be useful in detecting or diagnosing various immunological conditions related to expression of the respective antigens.

VI. Nucleic Acids

The described peptide sequences and the related reagents are useful in detecting, isolating, or identifying a DNA clone encoding HDTEA84, HSLJD37R, or RANKL, e.g., from a natural source. Typically, it will be useful in isolating a gene from mammal, and similar procedures will be applied to isolate genes from other species, e.g., warm blooded animals, such as birds and mammals. Cross hybridization will allow isolation of HDTEA84, HSLJD37R, or RANKL from other species. A number of different approaches should be available to successfully isolate a suitable nucleic acid clone.

The purified protein or defined peptides are useful for generating antibodies by standard methods, as described above. Synthetic peptides or purified protein can be presented to an immune system to generate monoclonal or polyclonal antibodies. See, e.g., Coligan (1991) Current Protocols in Immunology Wiley/Greene; and Harlow and Lane (1989) Antibodies: A Laboratory Manual, Cold Spring Harbor Press. Alternatively, the HDTEA84 can be used as a specific binding reagent, and advantage can be taken of its specificity of binding, much like an antibody would be used.

For example, the specific binding composition could be used for screening of an expression library made from a cell line which expresses an HDTEA84, HSLJD37R, or RANKL. The screening can be standard staining of surface expressed antigen constructs, or by panning. Screening of intracellular expression can also be performed by various staining or immunofluorescence procedures. The binding compositions could be used to affinity purify or sort out cells expressing the protein.

The peptide segments can also be used to predict appropriate oligonucleotides to screen a library. The genetic code can be used to select appropriate oligonucleotides useful as probes for screening. See, e.g., SEQ ID NO: 1, or 3, 5, or 7, and 12, 14, 16, or 18.-In combination with polymerase chain reaction (PCR) techniques, synthetic oligonucleotides will be useful in selecting correct clones from a library. Complementary sequences will also be used as probes, primers, or antisense strands. Based upon identification of the likely extracellular domain, various fragments should be particularly useful, e.g., coupled with anchored vector or poly-A complementary PCR techniques or with complementary DNA of other peptides.

This invention contemplates use of isolated DNA or fragments to encode a biologically active corresponding HDTEA84, HSLJD37R, or RANKL polypeptide. In addition, this invention covers isolated or recombinant DNA which encodes a biologically active protein or polypeptide which is capable of hybridizing under appropriate conditions with the DNA sequences described herein. Said biologically active protein or polypeptide can be an intact antigen, or fragment, and have an amino acid sequence disclosed in, e.g., SEQ ID NO: 2, 4, 6, 8, 13, 15, 17, or 19. Further, this invention covers the use of isolated or recombinant DNA, or fragments thereof, which-encode proteins which are homologous to a receptor or which was isolated using cDNA encoding a receptor as a probe. The isolated DNA can have the respective regulatory sequences in the 5′ and 3′ flanks, e.g., promoters, enhancers, poly-A addition signals, and others.

An “isolated” nucleic acid is a nucleic acid, e.g., an RNA, DNA, or a mixed polymer, which is substantially separated from other components which naturally accompany a native sequence, e.g., ribosomes, polymerases, and/or flanking genomic sequences from the originating species. The term embraces a nucleic acid sequence which has been removed from its naturally occurring environment, and includes recombinant or cloned DNA isolates and chemically synthesized analogs or analogs biologically synthesized by heterologous systems. A substantially pure molecule includes isolated forms of the molecule. Generally, the nucleic acid will be in a vector or fragment less than about 50 kb, usually less than about 30 kb, typically less than about 10 kb, and preferably less than about 6 kb.

An isolated nucleic acid will generally be a homogeneous composition of molecules, but will, in some embodiments, contain minor heterogeneity. This heterogeneity is typically found at the polymer ends or portions not critical to a desired biological function or activity.

A “recombinant” nucleic acid is defined either by its method of production or its structure. In reference to its method of production, e.g., a product made by a process, the process is use of recombinant nucleic acid techniques, e.g., involving human intervention in the nucleotide sequence, typically selection or production. Alternatively, it can be a nucleic acid made by generating a sequence comprising fusion of two fragments which are not naturally contiguous to each other, but is meant to exclude products of nature, e.g., naturally occurring mutants. Thus, e.g., products made by transforming cells with any unnaturally occurring vector is encompassed, as are nucleic acids comprising sequence derived using any synthetic oligonucleotide process. Such is often done to replace a codon with a redundant codon encoding the same or a conservative amino acid, while typically introducing or removing a sequence recognition site.

Alternatively, it is performed to join together nucleic acid segments of desired functions to generate a single genetic entity comprising a desired combination of functions not found in the commonly available natural forms. Restriction enzyme recognition sites are often the target of such artificial manipulations, but other site specific targets, e.g., promoters, DNA replication sites, regulation sequences, control sequences, or other useful features may be incorporated by design. A similar concept is intended for a recombinant, e.g., fusion, polypeptide. Specifically included are synthetic nucleic acids which, by genetic code redundancy, encode polypeptides similar to fragments of these antigens, and fusions of sequences from various different species variants.

A significant “fragment” in a nucleic acid context is a contiguous segment of at least about 17 nucleotides, generally at least about 22 nucleotides, ordinarily at least about 29 nucleotides, more often at least about 35 nucleotides, typically at least about 41 nucleotides, usually at least about 47 nucleotides, preferably at least about 55 nucleotides, and in particularly preferred embodiments will be at least about 60 or more nucleotides.

A DNA which codes for an HDTEA84, HSLJD37R, or RANKL protein will be particularly useful to identify genes, MRNA, and cDNA species which code for related or homologous proteins, as well as DNAs which code for homologous proteins from different species. There are likely homologs in other species, including primates, rodents, and birds. Various receptor proteins should be homologous and are encompassed herein. However, even genes encoding proteins that have a more distant evolutionary relationship to the antigen can readily be isolated under appropriate conditions using these sequences if they are sufficiently homologous. Primate HDTEA84, HSLJD37R, or RANKL proteins are of particular interest.

Recombinant clones derived from the genomic sequences, e.g., containing introns, will be useful for transgenic studies, including, e.g., transgenic cells and organisms, and for gene therapy. See, e.g., Goodnow (1992) “Transgenic Animals” in Roitt (ed.) Encyclopedia of Immunology, Academic Press, San Diego, pp. 1502-1504; Travis (1992) Science 256:1392-1394; Kuhn, et al. (1991) Science 254:707-710; Capecchi (1989) Science 244:1288; Robertson (1987 ed.) Teratocarcinomas and Embryonic Stem Cells: A Practical Approach, IRL Press, Oxford; and Rosenberg (1992) J. Clinical Oncology 10:180-199.

Substantial homology in the nucleic acid sequence comparison context means either that the segments, or their complementary strands, when compared, are identical when optimally aligned, with appropriate nucleotide insertions or deletions, in at least about 50% of the nucleotides, generally at least about 58%, ordinarily at least about 65%, often at least about 71%, typically at least about 77%, usually at least about 85%, preferably at least about 95 to 98% or more, and in particular embodiments, as high as about 99% or more of the nucleotides. Alternatively, substantial homology exists when the segments will hybridize under selective hybridization conditions, to a strand, or its complement, typically using a sequence of HDTEA84, e.g., in SEQ ID NO: 1. Typically, selective hybridization will occur when there is at least about 55% homology over a stretch of at least about 30 nucleotides, preferably at least about 75% over a stretch of about 25 nucleotides, and most preferably at least about 90% over about 20 nucleotides. See, Kanehisa (1984) Nuc. Acids Res. 12:203-213. The length of homology comparison, as described, may be over longer stretches, and in certain embodiments will be over a stretch of at least about 17 nucleotides, usually at least about 28 nucleotides, typically at least about 40 nucleotides, and preferably at least about 75 to 100 or more nucleotides.

Stringent conditions, in referring to homology in the hybridization context, will be stringent combined conditions of salt, temperature, organic solvents, and other parameters, typically those controlled in hybridization reactions. Stringent temperature conditions will usually include temperatures in excess of about 30° C., usually in excess of about 37° C., typically in excess of about 55° C., preferably in excess of about 70° C. Stringent salt conditions will ordinarily be less than about 1000 mM, usually less than about 400 mM, typically less than about 250 mM, preferably less than about 150 mM. However, the combination of parameters is much more important than the measure of any single parameter. See, e.g., Wetmur and Davidson (1968) J. Mol. Biol. 31:349-370. Hybridization under stringent conditions should give a background of at least 2-fold over background, preferably at least 3-5 or more.

HDTEA84, HSLJD37R, or RANKL from other mammalian species can be cloned and isolated by cross-species hybridization of closely related species. Homology may be relatively low between distantly related species, and thus hybridization of relatively closely related species is advisable. Alternatively, preparation of an antibody preparation which exhibits less species specificity may be useful in expression cloning approaches.

VII. Making Receptors; Mimetics

DNA which encodes the HDTEA84, HSLJD37R, or RANKL or fragments thereof can be obtained by chemical synthesis, screening cDNA libraries, or screening genomic libraries prepared from a wide variety of cell lines or tissue samples. See, e.g., Okayama and Berg (1982) Mol. Cell. Biol. 2:161-170; Gubler and Hoffman (1983) Gene 25:263-269; and Glover (ed. 1984) DNA Cloning: A Practical Approach, IRL Press, Oxford. Alternatively, the sequences provided herein provide useful PCR primers or allow synthetic or other preparation of suitable genes encoding a receptor; including, naturally occurring embodiments.

This DNA can be expressed in a wide variety of host cells for the synthesis of a full-length HDTEA84, HSLJD37R, or RANKL or fragments which can in turn, e.g., be used to generate polyclonal or monoclonal antibodies; for binding studies; for construction and expression of modified molecules; and for structure/function studies.

Vectors, as used herein, comprise plasmids, viruses, bacteriophage, integratable DNA fragments, and other vehicles which enable the integration of DNA fragments into the genome of the host. See, e.g., Pouwels, et al. (1985 and Supplements) Cloning Vectors: A Laboratory Manual, Elsevier, N.Y.; and Rodriguez, et al. (1988 eds.) Vectors: A Survey of Molecular Cloning Vectors and Their Uses, Buttersworth, Boston, Mass.

For purposes of this invention, DNA sequences are operably linked when they are functionally related to each other. For example, DNA for a presequence or secretory leader is operably linked to a polypeptide if it is expressed as a preprotein or participates in directing the polypeptide to the cell membrane or in secretion of the polypeptide. A promoter is operably linked to a coding sequence if it controls the transcription of the polypeptide; a ribosome binding site is operably linked to a coding sequence if it is positioned to permit translation. Usually, operably linked means contiguous and in reading frame, however, certain genetic elements such as repressor genes are not contiguously linked but still bind to operator sequences that in turn control expression. See e.g., Rodriguez, et al., Chapter 10, pp. 205-236; Balbas and Bolivar (1990) Methods in Enzymol. 185:14-37; and Ausubel, et al. (1993) Current Protocols in Molecular Biology, Greene and Wiley, NY.

Representative examples of suitable expression vectors include pCDNAl; pCD, see Okayama, et al. (1985) Mol. Cell Biol. 5:1136-1142; pMC1neo Poly-A, see Thomas, et al. (1987) Cell 51:503-512; and a baculovirus vector such as pAC 373 or pAC 610. See, e.g., Miller (1988) Ann. Rev. Microbiol. 42:177-199.

It will often be desired to express an HDTEA84, HSLJD37R, or RANKL polypeptide in a system which provides a specific or defined glycosylation pattern. See, e.g., Luckow and Summers (1988) Bio/Technology 6:47-55; and Kaufman (1990) Meth. Enzymol. 185:487-511. Preferred prokaryotic forms lack eukaryotic glycosylation patterns.

The HDTEA84, HSLJD37R, or RANKL, or a fragment thereof, may be engineered to be phosphatidyl inositol (PI) linked to a cell membrane, but can be removed from membranes by treatment with a phosphatidyl inositol cleaving enzyme, e.g., phosphatidyl inositol phospholipase-C. This releases the antigen in a biologically active form, and allows purification by standard procedures of protein chemistry. See, e.g., Low (1989) Biochim. Biophys. Acta 988:427-454; Tse, et al. (1985) Science 230:1003-1008; and Brunner, et al. (1991) J. Cell Biol. 114:1275-1283.

Now that the HDTEA84, HSLJD37R, and RANKL have been characterized, fragments or derivatives thereof can be prepared by conventional processes for synthesizing peptides. These include processes such as are described in Stewart and Young (1984) Solid Phase Peptide Synthesis, Pierce Chemical Co., Rockford, Ill.; Bodanszky and Bodanszky (1984) The Practice of Peptide Synthesis, Springer-Verlag, New York, N.Y.; Bodanszky (1984) The Principles of Peptide Synthesis, Springer-Verlag, New York; and Villafranca (ed. 1991) Techniques in Protein Chemistry II, Academic Press, San Diego, Calif.

VIII. Uses

The present invention provides reagents which will find use in diagnostic applications as described elsewhere herein, e.g., in the general description for T cell mediated conditions, or below in the description of kits for diagnosis. The genes will be useful in forensic analyses, e.g., to identify species, or to diagnose different cell subsets or types.

This invention also provides reagents with significant therapeutic value. The HDTEA84, HSLJD37R, or RANKL (naturally occurring or recombinant), fragments thereof, and antibodies thereto, along with compounds identified as having binding affinity to HDTEA84, HSLJD37R, or RANKL, should be useful in the treatment of conditions associated with abnormal physiology or development, including abnormal proliferation, e.g., cancerous conditions, or degenerative conditions. In particular, modulation of development of lymphoid cells will be achieved by appropriate therapeutic treatment using the compositions provided herein. For example, a disease or disorder associated with abnormal expression or abnormal signaling by an HDTEA84, HSLJD37R, or RANKL should be a likely target for an agonist or antagonist of the antigen. The antigen plays a role in regulation or development of hematopoietic cells, e.g., lymphoid cells, which affect immunological responses, e.g., autoimmune disorders.

In particular, the antigen may provide a costimulatory signal to cell activation, or be involved in regulation of cell proliferation or differentiation. Thus, the HDTEA84, HSLJD37R, or RANKL will likely modulate cells which possess a receptor therefor, e.g., T cell mediated interactions with other cell types. These interactions would lead, in particular contexts, to modulation of cell growth, cytokine synthesis by those or other cells, or development of particular effector cells.

Moreover, the HDTEA84, HSLJD37R, or RANKL or antagonists could redirect T cell responses, e.g., between Thl and Th2 polarization, or with ThO cells. Among these agonists should be various antibodies which recognize the appropriate epitopes, e.g., which mimic binding of HDTEA84, HSLJD37R, or RANKL to its receptor. Alternatively, they may bind to epitopes which sterically can block receptor binding. Bone morphogenesis may be regulated by these receptor segments.

HDTEA84, such as the naturally occurring secreted form of HDTEA84 or blocking antibodies, may also be useful. They may provide a selective and powerful way to modulate immune responses in abnormal situations, e.g., autoimmune disorders, including rheumatoid arthritis, systemic lupus erythematosis (SLE), Hashimoto's autoimmune thyroiditis, as well as acute and chronic inflammatory responses in which T cell activation, expansion, and/or immunological T cell memory play an important role. See also Samter, et al. (eds) Immunological Diseases vols. 1 and 2, Little, Brown and Co. Regulation of bone morphogenesis, T cell activation, expansion, and/or cytokine release by the naturally occurring secreted form of HDTEA84, HSLJD37R, or RANKL, or an antagonist thereof, may be effected.

In addition, certain combination compositions with other modulators of signaling would be useful. Such other signaling molecules might include, e.g., TCR reagents, CD40, CD40L, CTLA-8, CD28, SLAM, FAS, osteoprotegerin, and their respective antagonists, including antibodies.

Various abnormal conditions are known in each of the cell types shown to possess HDTEA84 MRNA by Northern blot analysis. See Berkow (ed.) The Merck Manual of Diagnosis and Therapy, Merck & Co., Rahway, N.J.; Thorn, et al. Harrison's Principles of Internal Medicine, McGraw-Hill, NY; and Weatherall, et al. (eds.) Oxford Textbook of Medicine, Oxford University Press, Oxford. Many other medical conditions and diseases involve T cells or are T cell mediated, and many of these may be responsive to treatment by an agonist or antagonist provided herein. See, e.g., Stites and Terr (eds; 1991) Basic and Clinical Immunology Appleton and Lange, Norwalk, Conn.; and Samter, et al. (eds) Immunological Diseases Little, Brown and Co. These problems should be susceptible to prevention or treatment using compositions provided herein.

HDTEA84, HSLJD37R, or RANKL antibodies can be purified and then administered to a patient, veterinary or human. These reagents can be combined for therapeutic use with additional active or inert ingredients, e.g., in conventional pharmaceutically acceptable carriers or diluents, e.g., immunogenic adjuvants, along with physiologically innocuous stabilizers, excipients, or preservatives. These combinations can be sterile filtered and placed into dosage forms as by lyophilization in dosage vials or storage in stabilized aqueous preparations. This invention also contemplates use of antibodies or binding fragments thereof, including forms which are not complement binding.

Drug screening using HDTEA84, HSLJD37R, or RANKL or fragments thereof can be performed to identify compounds having binding affinity to or other relevant biological effects on receptor functions, including isolation of associated components. Subsequent biological assays can then be utilized to determine if the compound has intrinsic stimulating activity or is a blocker or antagonist in that it blocks the activity of the antigen, e.g., mutein antagonists. Likewise, a compound having intrinsic stimulating activity can activate the signal pathway and is thus an agonist in that it overcome any blocking activity of these soluble forms of receptors. This invention further contemplates the therapeutic use of blocking antibodies to HDTEA84, HSLJD37R, or RANKL as agonists or antagonists and of stimulatory molecules, e.g., muteins, as agonists. This approach should be particularly useful with other soluble receptor species variants.

The quantities of reagents necessary for effective therapy will depend upon many different factors, including means of administration, target site, physiological state of the patient, and other medicants administered. Thus, treatment dosages should be titrated to optimize safety and efficacy. Typically, dosages used in vitro may provide useful guidance in the amounts useful for in situ administration of these reagents. Animal testing of effective doses for treatment of particular disorders will provide further predictive indication of human dosage. Various considerations are described, e.g., in Gilman, et al. (eds. 1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Penn. Methods for administration are discussed therein and below, e.g., for oral, intravenous, intraperitoneal, or intramuscular administration, transdermal diffusion, and others. Pharmaceutically acceptable carriers will include water, saline, buffers, and other compounds described, e.g., in the Merck Index, Merck & Co., Rahway, N.J. Dosage ranges would ordinarily be expected to be in amounts lower than 1 mM concentrations, typically less than about 10 μM concentrations, usually less than about 100 nM, preferably less than about 10 pM (picomolar), and most preferably less than about 1 fM (femtomolar), with an appropriate carrier. Slow release formulations, or a slow release apparatus will often be utilized for continuous or long term administration. See, e.g., Langer (1990) Science 249:1527-1533.

HDTEA84, HSLJD37R, or RANKL, fragments thereof, and antibodies to it or its fragments, antagonists, and agonists, may be administered directly to the host to be treated or, depending on the size of the compounds, it may be desirable to conjugate them to carrier proteins such as ovalbumin or serum albumin prior to their administration. Therapeutic formulations may be administered in many conventional dosage formulations. While it is possible for the active ingredient to be administered alone, it is preferable to present it as a pharmaceutical formulation. Formulations typically comprise at least one active ingredient, as defined above, together with one or more acceptable carriers thereof. Each carrier should be both pharmaceutically and physiologically acceptable in the sense of being compatible with the other ingredients and not injurious to the patient. Formulations include those suitable for oral, rectal, nasal, topical, or parenteral (including subcutaneous, intramuscular, intravenous and intradermal) administration. The formulations may conveniently be presented in unit dosage form and may be prepared by any methods well known in the art of pharmacy. See, e.g., Gilman, et al. (eds. 1990) Goodman and Gilman's: The Pharmacological Bases of Therapeutics, 8th Ed., Pergamon Press; and Remington's Pharmaceutical Sciences, 17th ed. (1990), Mack Publishing Co., Easton, Pa.; Avis, et al. (eds. 1993) Pharmaceutical Dosage Forms: Parenteral Medications, Dekker, New York; Lieberman, et al. (eds. 1990) Pharmaceutical Dosage Forms: Tablets, Dekker, New York; and Lieberman, et al. (eds. 1990) Pharmaceutical Dosage Forms: Disperse Systems, Dekker, New York. The therapy of this invention may be combined with or used in association with other agents, e.g., other modulators of cell activation, e.g., CD40, CD40 ligand, CD28, CTLA-4, B7, B70, SLAM, T cell receptor signaling entities, or their respective antagonists.

Both the naturally occurring and the recombinant forms of the HDTEA84, HSLJD37R, or RANKL of this invention are particularly useful in kits and assay methods which are capable of screening compounds for binding activity to the proteins. Several methods of automating assays have been developed in recent years so as to permit screening of tens of thousands of compounds in a short period. See, e.g., Fodor, et al. (1991) Science 251:767-773, which describes means for testing of binding affinity by a plurality of defined polymers synthesized on a solid substrate. The development of suitable assays can be greatly facilitated by the availability of large amounts of purified, soluble HDTEA84, HSLJD37R, or RANKL as provided by this invention.

Other methods can be used to determine the critical residues in the HDTEA84-ligand, HSLJD37R, or RANKL-ligand interactions. Mutational analysis can be performed, e.g., see Somoza, et al. (1993) J. Exp. Med. 178:549-558, to determine specific residues critical in the interaction and/or signaling. Both extracellular domains, involved in the soluble ligand interaction, or intracellular domain of a transmembrane form, which provides interactions important in intracellular signaling.

For example, antagonists can normally be found once the antigen has been structurally defined, e.g., by tertiary structure data. Testing of potential interacting analogs is now possible upon the development of highly automated assay methods using a purified HDTEA84, HSLJD37R, or RANKL. In particular, new agonists and antagonists will be discovered by using screening techniques described herein. Of particular importance are compounds found to have a combined binding affinity for a spectrum of HDTEA84 molecules, e.g., compounds which can serve as antagonists for species variants of HDTEA84, HSLJD37R, or RANKL.

One method of drug screening utilizes eukaryotic or prokaryotic host cells which are stably transformed with recombinant DNA molecules expressing an HDTEA84, HSLJD37R, or RANKL. Cells may be isolated which express an HDTEA84, HSLJD37R, or RANKL in isolation from other molecules. Such cells, either in viable or fixed form, can be used for standard binding partner binding assays. See also, Parce, et al. (1989) Science 246:243-247; and Owicki, et al. (1990) Proc. Nat'l Acad. Sci. USA 87:4007-4011, which describe sensitive methods to detect cellular responses.

Another technique for drug screening involves an approach which provides high throughput screening for compounds having suitable binding affinity to an HDTEA84, HSLJD37R, or RANKL and is described in detail in Geysen, European Patent Application 84/03564, published on September 13, 1984. First, large numbers of different small peptide test compounds are synthesized on a solid substrate, e.g., plastic pins or some other appropriate surface, see Fodor, et al. (1991). Then all the pins are reacted with solubilized, unpurified or solubilized, purified HDTEA84, HSLJD37R, or RANKL, and washed. The next step involves detecting bound HDTEA84, HSLJD37R, or RANKL.

Rational drug design may also be based upon structural studies of the molecular shapes of the HDTEA84, HSLJD37R, or RANKL and other effectors or analogs. Effectors may be other proteins which mediate other functions in response to binding, or other proteins which normally interact with HDTEA84, HSLJD37R, or RANKL. One means for determining which sites interact with specific other proteins is a physical structure determination, e.g., x-ray crystallography or 2 dimensional NMR techniques. These will provide guidance as to which amino acid residues form molecular contact regions. For a detailed description of protein structural determination, see, e.g., Blundell and Johnson (1976) Protein Crystallography, Academic Press, New York.

IX. Kits

This invention also contemplates use of HDTFA84, HSLJD37R, or RANKL proteins, fragments thereof, peptides, and their fusion products in a variety of diagnostic kits and methods for detecting, e.g., the presence of another HDTEA84, HSLJD37R, or RANKL or binding partner. Typically the kit will have a compartment containing either a defined HDTEA84, HSLJD37R, or RANKL peptide or gene segment or a reagent which recognizes one or the other, e.g., HDTEA84, HSLJD37R, or RANKL fragments or antibodies.

A kit for determining the binding affinity of a test compound to, e.g., an HDTEA84, would typically comprise a test compound; a labeled compound, for example a binding partner or antibody having known binding affinity for HDTFA84; a source of HDTEA84 (naturally occurring or recombinant); and a means for separating bound from free labeled compound, such as a solid phase for immobilizing the molecule. Once compounds are screened, those having suitable binding affinity to the antigen can be evaluated in suitable biological assays, as are well known in the art, to determine whether they act as agonists or antagonists to the HDTEA84 signaling pathway. The availability of recombinant HDTEA84 polypeptides also provide well defined standards for calibrating such assays.

A preferred kit for determining the concentration of, e.g., an HDTEA84 in a sample would typically comprise a labeled compound, e.g., binding partner or antibody, having known binding affinity for the antigen, a source of antigen (naturally occurring or recombinant) and a means for separating the bound from free labeled compound, e.g., a solid phase for immobilizing the HDTEA84. Compartments containing reagents, and instructions, will normally be provided.

Antibodies, including antigen binding fragments, specific for, e.g., the HDTEA84 or fragments, are useful in diagnostic applications to detect the presence of elevated levels of HDTEA84 and/or its fragments. Such diagnostic assays can employ lysates, live cells, fixed cells, immunofluorescence, cell cultures, body fluids, and further can involve the detection of antigens related to the antigen in serum, or the like. Diagnostic assays may be homogeneous (without a separation step between free reagent and antigen-binding partner complex) or heterogeneous (with a separation step). Various commercial assays exist, such as radioimmunoassay (RIA), enzyme-linked immunosorbent assay (ELISA), enzyme immunoassay (EIA), enzyme-multiplied immunoassay technique (EMIT), substrate-labeled fluorescent immunoassay (SLFIA), and the like. See, e.g., Van Vunakis, et al. (1980) Meth Enzymol. 70:1-525; Harlow and Lane (1980) Antibodies: A Laboratory Manual, CSH Press, NY; and Coligan, et al. (eds. 1993) Current Protocols in Immunology, Greene and Wiley, NY.

Anti-idiotypic antibodies may have similar use to diagnose presence of antibodies against an HDTEA84, HSLJD37R, or RANKL, as such may be diagnostic of various abnormal states. For example, overproduction of HDTEA84, HSLJD37R, or RANKL may result in production of various immunological reactions which may be diagnostic of abnormal physiological states, particularly in proliferative cell conditions such as cancer or abnormal activation or differentiation.

Frequently, the reagents for diagnostic assays are supplied in kits, so as to optimize the sensitivity of the assay. For the subject invention, depending upon the nature of the assay, the protocol, and the label, either labeled or unlabeled antibody or binding partner, or labeled HDTEA84 is provided. This is usually in conjunction with other additives, such as buffers, stabilizers, materials necessary for signal production such as substrates for enzymes, and the like. Preferably, the kit will also contain instructions for proper use and disposal of the contents after use. Typically the kit has compartments for each useful reagent. Desirably, the reagents are provided as a dry lyophilized powder, where the reagents may be reconstituted in an aqueous medium providing appropriate concentrations of reagents for performing the assay.

Many of the aforementioned constituents of the drug screening and the diagnostic assays may be used without modification or may be modified in a variety of ways. For example, labeling may be achieved by covalently or non-covalently joining a moiety which directly or indirectly provides a detectable signal. In any of these assays, the binding partner, test compound, HDTEA84, or antibodies thereto can be labeled either directly or indirectly. Possibilities for direct labeling include label groups: radiolabels such as 125_{I}, enzymes (U.S. Pat. No. 3,645,090) such as peroxidase and alkaline phosphatase, and fluorescent labels (U.S. Pat. No. 3,940,475) capable of monitoring the change in fluorescence intensity, wavelength shift, or fluorescence polarization. Possibilities for indirect labeling include biotinylation of one constituent followed by binding to avidin coupled to one of the above label groups.

There are also numerous methods of separating the bound from the free HDTEA84, HSLJD37R, or RANKL, or alternatively the bound from the free test compound. The HDTEA84, HSLJD37R, or RANKL can be immobilized on various matrixes followed by washing. Suitable matrixes include plastic such as an ELISA plate, filters, and beads. See, e.g., Coligan, et al. (eds. 1993) Current Protocols in Immunology, Vol. 1, Chapter 2, Greene and Wiley, NY. Other suitable separation techniques include, without limitation, the fluorescein antibody magnetizable particle method described in Rattle, et al. (1984) Clin. Chem. 30:1457-1461, and the double antibody magnetic particle separation as described in U.S. Pat. No. 4,659,678.

Methods for linking proteins or their fragments to the various labels have been extensively reported in the literature and do not require detailed discussion here. Many of the techniques involve the use of activated carboxyl groups either through the use of carbodiimide or active esters to form peptide bonds, the formation of thioethers by reaction of a mercapto group with an activated halogen such as chloroacetyl, or an activated olefin such as maleimide, for linkage, or the like. Fusion proteins will also find use in these applications.

Another diagnostic aspect of this invention involves use of oligonucleotide or polynucleotide sequences taken from the sequence of an HDTEA84, HSLJD37R, or RANKL. These sequences can be used as probes for detecting levels of the HDTEA84, HSLJD37R, or RANKL message in samples from patients suspected of having an abnormal condition, e.g., cancer or developmental problem. Since, e.g., the RANKL, antigen is a marker for activation, it may be useful to determine the numbers of activated T cells to determine, e.g., when additional suppression may be called for. The preparation of both RNA and DNA nucleotide sequences, the labeling of the sequences, and the preferred size of the sequences has received ample description and discussion in the literature. See, e.g., Langer-Safer, et al. (1982) Proc. Nat'l. Acad. Sci. 79:4381-4385; Caskey (1987) Science 236:962-967; and Wilchek et al. (1988) Anal. Biochem. 171:1-32.

Diagnostic kits which also test for the qualitative or quantitative presence of other markers are also contemplated. Diagnosis or prognosis may depend on the combination of multiple indications used as markers. Thus, kits may test for combinations of markers. See, e.g., Viallet, et al. (1989) Progress in Growth Factor Res. 1:89-97. Other kits may be used to evaluate T cell subsets.

X. Methods for Isolating TNF-R Specific Binding Partners

The HDTEA84, HSLJD37R, or RANKL protein should interact with a TNF ligand based, e.g., upon its similarity in structure and function to other cell surface antigens exhibiting similar structure and cell type specificity of expression. Methods to isolate a ligand are made available by the ability to make purified HDTEA84, HSLJD37R, or RANKL for screening programs. Sequences provided herein will allow for screening or isolation of specific ligands. Many methods exist for expression cloning, panning, affinity isolation, or other means to identify a ligand. A two-hybrid selection system may also be applied making appropriate constructs with the available HDTEA84, HSLJD37R, or RANKL sequences. See, e.g., Fields and Song (1989) Nature 340:245-246.

The broad scope of this invention is best understood with reference to the following examples, which are not intended to limit the invention to specific embodiments.

### EXAMPLES

General Methods

Some of the standard methods are described or referenced, e.g., in Maniatis, et al. (1982) Molecular Cloning, A Laboratory Manual, Cold Spring Harbor Laboratory, Cold Spring Harbor Press; Sambrook, et al. (1989) Molecular Cloning: A Laboratory Manual (2d ed.), vols. 1-3, CSH Press, NY; Ausubel, et al., Biology, Greene Publishing Associates, Brooklyn, N.Y.; or Ausubel, et al. (1987 and Supplements) Current Protocols in Molecular Biology, Greene and Wiley, New York; Innis, et al. (eds. 1990) PCR Protocols: A Guide to Methods and Applications, Academic Press, N.Y. Methods for protein purification include such methods as ammonium sulfate precipitation, column chromatography, electrophoresis, centrifugation, crystallization, and others. See, e.g., Ausubel, et al. (1987 and periodic supplements); Deutscher (1990) “Guide to Protein Purification” in Methods in Enzymol. vol. 182, and other volumes in this series; and manufacturer's literature on use of protein purification products, e.g., Pharmacia, Piscataway, N.J., or Bio-Rad, Richmond, Calif. Combination with recombinant techniques allow fusion to appropriate segments, e.g., to a FLAG sequence or an equivalent which can be fused via a protease-removable sequence. See, e.g., Hochuli (1989) Chemische Industrie 12:69-70; Hochuli (1990) “Purification of Recombinant Proteins with Metal Chelate Absorbent” in Setlow (ed.) Genetic Engineering, Principle and Methods 12:87-98, Plenum Press, N.Y.; and Crowe, et al. (1992) QIAexpress: The High Level Expression & Protein Purification System QIAGEN, Inc., Chatsworth, Calif. Cell culture techniques are described in Doyle, et al. (eds. 1994) Cell and Tissue Culture: Laboratory Procedures, John Wiley and Sons, NY.

Standard immunological techniques are described, e.g., in Hertzenberg, et al. (eds. 1996) Weir's Handbook of Experimental Immunology vols. 1-4, Blackwell Science; Coligan (1991) Current Protocols in Immunology Wiley/Greene, NY; and Methods in Enzymology volumes. 70, 73, 74, 84, 92, 93, 108, 116, 121, 132, 150, 162, and 163.

FACS analyses are described in Melamed, et al. (1990) Flow Cytometry and Sorting Wiley-Liss, Inc., New York, NY; Shapiro (1988) Practical Flow Cytometry Liss, New York, N.Y.; and Robinson, et al. (1993) Handbook of Flow Cytometry Methods Wiley-Liss, New York, N.Y. Fluorescent labeling of appropriate reagents was performed by standard methods.

### Example 1

### Cloning of Soluble TNF-R

The HDTEA84 was assembled by careful analysis of ESTs found in various databases. These ESTs were from CDNA libraries derived from Hodgkin's lymphoma, endothelial cells, keratinocytes, prostate, and cerebellum. PCR primers are designed and synthesized and a PCR product is obtained from any of these libraries. This product is used as a hybridization clone to screen these libraries for a full length clone, which may include a transmembrane segment.

Likewise, the HSLJD37R was identified from sequences derived from cDNA libraries from: smooth muscle, pancreas tumor, adipocytes, HUVEC cells, adult pulmonary, endothelial cells, prostate cell line PC3, microvascular endothelial cells, fetal heart, and dendritic cells. A Genbank report by Pan, et al. has been submitted. See GenBank Accession 3549263. Other sequences were detected in libraries from: multiple sclerosis lesions, breast, kidney, and germinal center B cells. RT-PCT showed signal in B clels, PBL, granulocytes, T cells, monocytes, dendritic cell subpopulations including PMA/ionomycin treated, U937 cells, JY cells, MRC5 cells, CHA, Jurkat, and YCl cells. This suggests that the transcript is widely expressed.

RANKL was also identified in cDNA libraries from specific tissues, as described.

### Example 2

### Cellular Expression of TNF receptors

A probe specific for cDNA encoding the HDTEA84, HSLJD37R, or RANKL is used to determine tissue distribution of message encoding the antigen. Standard hybridization probes may be used to do a Northern analysis of RNA from appropriate sources, either cells, e.g., stimulated, or in various physiological states, in various tissues, e.g., spleen, liver, thymus, lung, etc., or in various species. Southern analysis of cDNA libraries may also provide valuable distribution information. Standard tissue blots or species blots are commercially available. Similar techniques will be useful for evaluating diagnostic or medical conditions which may correlate with expression in various cell types.

PCR analysis using appropriate primers may also be used. Antibody analysis, including immunohistochemistry or FACS, may be used to determine cellular or tissue distribution.

Southern blot analysis of primate cDNA libraries is performed on, e.g.,: U937 premonocytic line, resting (M100); elutriated monocytes, activated with LPS, IFNY, anti-IL-10 for 4, 16 h pooled (M106); elutriated monocytes, activated with LPS, IFNγ, IL-10 for 4, 16 h pooled (M107); elutriated monocytes, activated LPS for 1 h (M108); elutriated monocytes, activated LPS for 6 h (M109); dendritic cells (DC) 30% CD1a+, from CD34+ GM-CSF, TNFα 12 days, resting; DC 70% CD1a+, from CD34+ GM-CSF, TNFα 12 days, resting (D101); DC 70% CDla+, from CD34+ GM-CSF, TNFα 12 days, activated with PMA and ionomycin for 1 hr (D102); DC 70% CDla+, from CD34+ GM-CSF, TNFα 12 days, activated with PMA and ionomycin for 6 hr (D103); DC 95% CD1a+, from CD34+ GM-CSF, TNFα 12 days activated with PMA and ionomycin for 1 or 6 hr, pooled; DC from monocytes GM-CSF, IL-4 5 days, resting (D107); DC from monocytes GM-CSF, IL-4 5 days, resting (D108); DC from monocytes GM-CSF, IL-4 5 days, activated TNFα, monocyte supe for 4, 16 h pooled (D110); EBV transfected B cell lines, resting; spleenocytes, resting; spleenocytes, activated with PMA and ionomycin; 20 NK clones resting, pooled; 20 NK clones activated with PMA and ionomycin, pooled; NKL clone, IL-2 treated; NK cytotoxic clone, resting; adipose tissue fetal 28 wk male (0108); brain fetal 28 wk male (0104); gallbladder fetal 28 wk male (0106); heart fetal 28 wk male (0103); small intestine fetal 28 wk male (0107); kidney fetal 28 wk male (0100); liver fetal 28 wk male (0102); lung fetal 28 wk male (0101); ovary fetal 25 wk female (0109); adult placenta 28 wk (0113); spleen fetal 28 wk male (0112); testes fetal 28 wk male (0111); uterus fetal 25 wk female (0110); THO clone Mot 72, resting (T102); T cell, THO clone Mot 72, activated with anti-CD28 and anti-CD3 for 3, 6, 12 h pooled (T103); T cell, TH0 clone Mot 72, anergic treated with specific peptide for 2, 7, 12 h pooled (T104); ThO subtraction of resting from activated; T cell, TH1 clone HY06, resting (T107); T cell, TH1 clone HY06, activated with anti-CD28 and anti-CD3 for 3, 6, 12 h pooled (T108); T cell, THI clone HY06, anergic treated with specific peptide for 2, 6, 12 h pooled (T109); Thl subtraction of resting from activated; T cell, TH2 clone HY935, resting (T110); T cell, TH2 clone HY935, activated with anti-CD28 and anti-CD3 for 2, 7, 12 h pooled (T111); and Th2 subtraction of resting from activated.

Samples for mouse mRNA distribution may include, e.g.,: resting mouse fibroblastic L cell line (C200); Braf:ER (Braf fusion to estrogen receptor) transfected cells, control (C201); T cells, TH1 polarized (Mel14 bright, CD4+ cells from spleen, polarized for 7 days with IFN-γ and anti IL-4; T200); T cells, TH2 polarized (Mel14 bright, CD4+ cells from spleen, polarized for 7 days with IL-4 and anti-IFN-γ; T201); T cells, highly TH1 polarized (see Openshaw, et al. (1995) J. Exp. Med. 182:1357-1367; activated with anti-CD3 for 2, 6, 16 h pooled; T202); T cells, highly TH2 polarized (see Openshaw, et al. (1995) J. Exp. Med. 182:1357-1367; activated with anti-CD3 for 2, 6, 16 h pooled; T203); CD44− CD25+ pre T cells, sorted from thymus (T204); TH1 T cell clone D1.1, resting for 3 weeks after last stimulation with antigen (T205); TH1 T cell clone D1.1, 10 μg/ml ConA stimulated 15 h (T206); TH2 T cell clone CDC35, resting for 3 weeks after last stimulation with antigen (T207); TH2 T cell clone CDC35, 10 μg/ml ConA stimulated 15 h (T208); Mel 14+naive T cells from spleen, resting (T209); Mell4+T cells, polarized to Th1 with IFN-γ/IL-12/anti-IL-4 for 6, 12, 24 h pooled (T210); Mel14+T cells, polarized to Th2 with IL-4/anti-IFN-γ for 6, 13, 24 h pooled (T211); unstimulated mature B cell leukemia cell line A20 (B200); unstimulated B cell line CH12 (B201); unstimulated large B cells from spleen (B202); B cells from total spleen, LPS activated (B203); metrizamide enriched dendritic cells from spleen, resting (D200); dendritic cells from bone-marrow, resting (D201); monocyte cell line RAW 264.7 activated with LPS 4 h (M200); bone-marrow macrophages derived with GM and M-CSF (M201); macrophage cell line J774, resting (M202); macrophage cell line J774+LPS +anti-IL-10 at 0.5, 1, 3, 6, 12 h pooled (M203); macrophage cell line J774+LPS +IL-10 at 0.5, 1, 3, 5, 12 h pooled (M204); aerosol challenged mouse lung tissue, Th2 primers, aerosol OVA challenge 7, 14, 23 h pooled (see Garlisi, et al. (1995) Clinical Immunology and Immunopathology 75:75-83; X206); Nippostrongulus-infected lung tissue (see Coffman, et al. (1989) Science 245:308-310;×200); total adult lung, normal (0200); total lung, rag-l (see Schwarz, et al. (1993) Immunodeficiency 4:249-252; 0205); IL-10 K.O. spleen (see Kuhn, et al. (1991) Cell 75:263-274;×201); total adult spleen, normal (0201); total spleen, rag-1 (0207); IL-10 K.O. Peyer's patches (0202); total Peyer's patches, normal (0210); IL-10 K.O. mesenteric lymph nodes (X203); total mesenteric lymph nodes, normal (0211); IL-10 K.O. colon (X203); total colon, normal (0212); NOD mouse pancreas (see Makino, et al. (1980) Jikken Dobutsu 29:1-13;×205); total thymus, rag-1 (0208); total kidney, rag-1 (0209); total heart, rag-1 (0202); total brain, rag-1 (0203); total testes, rag-1 (0204); total liver, rag-1 (0206); rat normal joint tissue (0300); and rat arthritic joint tissue (X300).

### Example 3

### Purification of TNF Receptor Protein

Multiple transfected cell lines are screened for one which expresses the antigen, membrane bound or soluble forms, at a high level compared with other cells. Various cell lines are screened and selected for their favorable properties in handling. Natural receptors can be isolated from natural sources, or by expression from a transformed cell using an appropriate expression vector. Purification of the expressed protein is achieved by standard procedures, or may be combined with engineered means for effective purification at high efficiency from cell lysates or supernatants. FLAG or His₆ segments can be used for such purification features.

### Example 4

### Isolation of Homologous Receptor Genes

The primate HDTEA84, HSLJD37R, or RANKL cDNA can be used as a hybridization probe to screen a library from a desired source, e.g., a primate cell cDNA library. Many different species can be screened both for stringency necessary for easy hybridization, and for presence using a probe. Appropriate hybridization conditions will be used to select for clones exhibiting specificity of cross hybridization.

Screening by hybridization or PCR using degenerate probes based upon the peptide sequences will also allow isolation of appropriate clones. Alternatively, use of appropriate primers for PCR screening will yield enrichment of appropriate nucleic acid clones.

Similar methods are applicable to isolate either species, polymorphic, or allelic variants. Species variants are isolated using cross-species hybridization techniques based upon isolation of a full length isolate or fragment from one species as a probe.

Alternatively, antibodies raised against human HDTEA84 will be used to screen for cells which express cross-reactive proteins from an appropriate, e.g., cDNA library. The purified protein or defined peptides are useful for generating antibodies by standard methods, as described above. Synthetic peptides or purified protein are presented to an immune system to generate monoclonal or polyclonal antibodies. See, e.g., Coligan (1991) Current Protocols in Immunology Wiley/Greene; and Harlow and Lane (1989) Antibodies: A Laboratory Manual Cold Spring Harbor Press. The resulting antibodies are used, e.g., for screening, panning, or sorting.

### Example 5

### Preparation of Antibodies

Synthetic peptides or purified protein, natural or recombinant, are presented to an immune system to generate monoclonal or polyclonal antibodies. See, e.g., Coligan (1991) Current Protocols in Immunology Wiley/Greene; and Harlow and Lane (1989) Antibodies: A Laboratory Manual Cold Spring Harbor Press. Polyclonal serum, or hybridomas may be prepared. In appropriate situations, the binding reagent is either labeled as described above, e.g., fluorescence or otherwise, or immobilized to a substrate for panning methods.

### Example 6

### Isolation of Ligand for Receptor

A construct for expression of the product can be used as a specific binding reagent to identify its binding partner, e.g., ligand, by taking advantage of its specificity of binding, much like an antibody would be used. A receptor reagent is either labeled as described above, e.g., fluorescence or otherwise, or immobilized to a substrate for panning methods. See also Anderson, et al. (1997) Nature 390:175-179, which is incorporated herein by reference.

The binding composition is used to screen an expression library made from a cell line which expresses a binding partner, i.e., TNF family ligand. Standard staining techniques are used to detect or sort intracellular or surface expressed receptor, or surface expressing transformed cells are screened by panning. Screening of intracellular expression is performed by various staining or immunofluorescence procedures. See also McMahan, et al. (1991) EMBO J. 10:2821-2832.

Alternatively, receptor reagents are used to affinity purify or sort out cells expressing a receptor. See, e.g., Sambrook, et al. or Ausubel, et al.

Another strategy is to screen for a membrane bound ligand by panning. The cDNA containing ligand cDNA is constructed as described above. The ligand can be immobilized and used to immobilize expressing cells. Immobilization may be achieved by use of appropriate antibodies which recognize, e.g., a FLAG sequence or a receptor fusion construct, or by use of antibodies raised against the first antibodies. Recursive cycles of selection and amplification lead to enrichment of appropriate clones and eventual isolation of ligand expressing clones.

Phage expression libraries can be screened by receptor. Appropriate label techniques, e.g., anti-FLAG antibodies, will allow specific labeling of appropriate clones.

### Example 7

### Chromosomal Mapping

The receptor genes can be mapped to the primate chromosome. A BIOS Laboratories (New Haven, CT) mouse somatic cell hybrid panel can be combined with PCR.

Chromosome spreads are prepared. In situ hybridization is performed on chromosome preparations obtained from phytohemagglutinin-stimulated human lymphocytes cultured for 72 h. 5-bromodeoxyuridine is added for the final seven hours of culture (60 μg/ml of medium), to ensure a posthybridization chromosomal banding of good quality.

A PCR fragment, amplified with the help of primers, is cloned into an appropriate vector. The vector is labeled by nick-translation with ³H. The radiolabeled probe is hybridized to metaphase spreads at final concentration of 200 ng/ml of hybridization solution as described in Mattei, et al. (1985) Hum. Genet. 69:327-331.

After coating with nuclear track emulsion (KODAK NTB₂), slides are exposed. To avoid any slipping of silver grains during the banding procedure, chromosome spreads are first stained with buffered Giemsa solution and metaphase photographed. R-banding is then performed by the fluorochrome-photolysis-Giemsa (FPG) method and metaphases rephotographed before analysis.

All citations herein are incorporated herein by reference to the same extent as if each individual publication or patent application was specifically and individually indicated to be incorporated by reference.

Many modifications and variations of this invention can be made without departing from its spirit and scope, as will be apparent to those skilled in the art. The specific embodiments described herein are offered by way of example only, and the invention is to be limited by the terms of the appended claims, along with the full scope of equivalents to which such claims are entitled.

What is claimed is:

1. An isolated or recombinant polynucleotide encoding an antigenic polypeptide comprising:
a) at least 17 contiguous amino acids from the mature polypeptide from SEQ ID NO: 2;
b) at least 17 contiguous amino acids from the mature polypeptide from SEQ ID NO: 4:
c) at least 17 contiguous amino acids from the mature polypeptide from SEQ ID NO: 6:
d) at least 17 contiguous amino acids from the mature polypeptide from SEQ ID NO: 8:
e) at least 17 contiguous amino acids from the mature polypeptide from SEQ ID NO: 13:
f) at least 17 contiguous amino acids from the polypeptide from SEQ ID NO: 15:
g) at least 17 contiguous amino acids from the polypeptide from SEQ ID NO: 17: or
h) at least 17 contiguous amino acids from the polypeptide from SEQ ID NO: 19.

2. The polynucleotide of claim 1, encoding all of the polypeptide of:
a) mature SEQ ID NO: 2;
b) mature SEQ ID NO: 4;
c) mature SEQ ID NO: 6;
d) mature SEQ ID NO: 8;
e) mature SEQ ID NO: 13;
f) SEQ ID NO: 15;
g) SEQ ID NO: 17; or
h) SEQ ID NO: 19.

3. The polynucleotide of claim 1, which hybridizes at 55° C., less than 500 mM salt, and 50% formamide to:
a) the mature polypeptide coding portion of SEQ ID NO: 1;
b) the mature polypeptide coding portion of SEQ ID NO: 3;
c) the mature polypeptide coding portion of SEQ ID NO: 5;
d) the mature polypeptide coding portion of SEQ ID NO: 7;
e) the mature polypeptide coding portion of SEQ ID NO: 12;
f) the polypeptide coding portion of SEQ ID NO: 14;
g) the polypeptide coding portion of SEQ ID NO: 16;or
h) the polypeptide coding portion of SEQ ID NO: 18.

4. The polynucleotide of claim 3, comprising:
a) at least 35 contiguous nucleotides of the mature coding portion of SEQ ID NO: 1;
b) at least 35 contiguous nucleotides of the mature coding portion of SEQ ID NO: 3;
c) at least 35 contiguous nucleotides of the mature coding portion of SEQ ID NO: 5;
d) at least 35 contiguous nucleotides of the mature coding portion of SEQ ID NO: 7;
e) at least 35 contiguous nucleotides of the mature coding portion of SEQ ID NO: 12;
f) at least 35 contiguous nucleotides of the coding portion of SEQ ID NO: 14;
g) at least 35 contiguous nucleotides of the coding portion of SEQ ID NO: 16; or
h) at least 35 contiguous nucleotides of the coding portion of SEQ ID NO: 18.

5. An expression vector comprising the polynucleotide of claim 1.

6. A host cell containing the expression vector of claim 5, including a eukaryotic cell.

7. A method of making an antigenic polypeptide comprising expressing a recombinant polynucleotide of claim 1.

8. A method for detecting a polynucleotide of claim 1, comprising contacting said polynucleotide with a probe that hybridizes, under stringent conditions, to at least 25 contiguous nucleotides of:
a) the polynucleotide comprising the signal processed coding portion of SEQ ID NO: 1;
b) the polynucleotide comprising the signal processed coding portion of SEQ ID NO: 3;
c) the polynucleotide comprising the signal processed coding portion of SEQ ID NO: 5;
d) the polynucleotide comprising the signal processed coding portion of SEQ ID NO: 7;
e) the polynucleotide comprising the signal processed coding portion of SEQ ID NO: 12;
f) the polynucleotide comprising the coding portion of SEQ ID NO: 14;
g) the polynucleotide comprising the coding portion of SEQ ID NO: 16; or
h) the polynucleotide comprising the coding portion of SEQ ID NO: 18;
to form a duplex, wherein detection of said duplex indicates the presence of said polynucleotide.

9. A kit for the detection of a polynucleotide of claim 1, comprising a compartment containing a probe that hybridizes, under stringent hybridization conditions, to at least 17 contiguous nucleotides of a polynucleotide of claim 1 to form a duplex.

10. The kit of claim 9, wherein said probe is detectably labeled.

11. A binding compound comprising an antibody binding site which specifically binds to:
a) at least 17 contiguous amino acids from the signal processed form of SEQ ID NO: 2;
b) at least 17 contiguous amino acids from the signal processed form of SEQ ID NO: 4;
c) at least 17 contiguous amino acids from the signal processed form of SEQ ID NO: 6;
d) at least 17 contiguous amino acids from the signal processed form of SEQ ID NO: 8;
e) at least 17 contiguous amino acids from the signal processed form of SEQ ID NO: 13;
f) at least 17 contiguous amino acids from SEQ ID NO: 15;
g) at least 17 contiguous amino acids from SEQ ID NO: 17; or
h) at least 17 contiguous amino acids from SEQ ID NO: 19.

12. The binding compound of claim 11, wherein:
a) said antibody binding site is:
1) selectively immunoreactive with a polypeptide of the signal processed form of SEQ ID NO: 2;
2) selectively immunoreactive with a polypeptide of the signal processed form of SEQ ID NO: 4;
3) selectively immunoreactive with a polypeptide of the signal processed form of SEQ ID NO: 6;
4) selectively immunoreactive with a polypeptide of the signal processed form of SEQ ID NO: 8;
5) selectively immunoreactive with a polypeptide of the signal processed form of SEQ ID NO: 13;
6) selectively immunoreactive with a polypeptide of SEQ ID NO: 15;
7) selectively immunoreactive with a polypeptide of SEQ ID NO: 17;
8) selectively immunoreactive with a polypeptide of SEQ ID NO: 19; or
b) said binding compound is:
1) an antibody molecule;
2) a polyclonal antiserum;
3) detectably labeled;
4) sterile; or
5) in a buffered composition.

13. A method using the binding compound of claim 11, comprising contacting said binding compound with a biological sample comprising an antigen, thereby forming a binding compound:antigen complex.

14. The method of claim 13, wherein said biological sample is from a human, and wherein said binding compound is an antibody.

15. A detection kit comprising said binding compound of claim 12, and:
a) instructional material for the use of said binding compound for said detection; or b) a compartment providing segregation of said binding compound.

16. A substantially pure or isolated antigenic polypeptide, which binds to said binding composition of claim 11, and further comprises at least 17 contiguous amino acids from:
a) the signal processed polypeptide from SEQ ID NO: 2;
b) the signal processed polypeptide from SEQ ID NO: 4;
c) the signal processed polypeptide from SEQ ID NO: 6;
d) the signal processed polypeptide from SEQ ID NO: 8;
e) the signal processed polypeptide from SEQ ID NO: 13;
f) SEQ ID NO: 15;
g) SEQ ID NO: 17; or
h) SEQ ID NO: 19.

17. The polypeptide of claim 16, which:
a) comprises at least a fragment of at least 25 contiguous amino acid residues from a signal processed primate HDTEA84 protein;
b) comprises at least a fragment of at least 25 contiguous amino acid residues from a signal processed primate HSLJD37R protein;
c) comprises at least a fragment of at least 25 contiguous amino acid residues from a signal processed rodent RANKL protein; or
d) comprises at least a fragment of at least 25 contiguous amino acid residues from primate RANKL protein;
e) is a soluble polypeptide;
f) is detectably labeled;
g) is in a sterile composition;
h) is in a buffered composition;
i) binds to an sialic acid residue;
j) is recombinantly produced, or
k) has a naturally occurring polypeptide sequence.

18. The polypeptide of claim 17, which:
a) comprises at least 17 contiguous amino acids of the signal processed SEQ ID NO: 2;
b) comprises at least 17 contiguous amino acids of the signal processed SEQ ID NO: 4;
c) comprises at least 17 contiguous amino acids of the signal processed SEQ ID NO: 6;
d) comprises at least 17 contiguous amino acids of the signal processed SEQ ID NO: 8;
e) comprises at least 17 contiguous amino acids of the signal processed SEQ ID NO: 13;
f) comprises at least 17 contiguous amino acids of SEQ ID NO: 15;
g) comprises at least 17 contiguous amino acids of SEQ ID NO: 17; or
h) comprises at least 17 contiguous amino acids of SEQ ID NO: 19.

19. A method of modulating a precursor cell physiology or function comprising a step of contacting said cell with:
a) a binding compound which binds to said polypeptide of claim 16;
b) an HDTEA84 polypeptide;
c) an HSLJD37R polypeptide; or
d) a RANKL polypeptide.

20. The method of claim 19, wherein said contacting is in combination with a TNF family ligand, or an antagonist of said TNF family ligand.
